(19) United States
(12) Patent Application Publication
(10) Pub. No.: US 2009/0270312 A1
(43) Pub. Date: Oct. 29, 2009

(54) HIV-I GP41 FUSION PEPTIDES FOR IMMUNOMODULALTION

(76) Inventors: Yechiel Shai, Yehud (IL); Irun R. Cohen, Rehovot (IL); Francisco J. Quintana, Capital Federal (AR); Doron Gerber, Herzliya (IL)

Correspondence Address: FENNEMORE CRAIG, 3003 NORTH CENTRAL AVENUE, SUITE 2600, PHOENIX, AZ, 85012, US

(21) Appl. No.: 11/722,064

(22) PCT Filed: Jan. 24, 2006
(86) PCT No.: PCT/IL2006/000096

§ 371 Date: Nov. 12, 2007

Related U.S. Application Data
(60) Provisional application No. 60/645,605, filed on Jan. 24, 2005.

(51) Int. Cl. A61K 38/16; A61K 38/08; C07K 14/00; C07K 7/00

(52) U.S. Cl. 514/12; 514/16; 514/15; 530/324; 530/328

(57) ABSTRACT

The present invention provides pharmaceutical compositions and methods for prevention or treatment of autoimmune diseases and other T cell mediated inflammatory diseases and conditions, which comprise as an active ingredient an effective quantity of a peptide derived from HIV gp41 fusion peptide domain or fragments, analogs, homologs and derivatives thereof. The invention further provides novel peptides derived from HIV gp41 fusion peptide domain, useful in the treatment of T cell mediated pathologies.

### FIELD OF THE INVENTION

The present invention provides novel uses of peptides derived from the HIV gp41 fusion peptide domain, in methods for prevention or treatment of autoimmune and other T cell-mediated pathologies which comprise administering to a subject an effective quantity of an HIV gp41 fusion peptide or fragments, homologs and derivatives thereof. Certain novel fragments of the HIV gp41 fusion peptide useful in the methods of the present invention are claimed as such.

### BACKGROUND OF THE INVENTION

Human immunodeficiency virus (HIV) infection confounds the immune response. Untreated HIV infection usually leads to a state of general immunosuppression, the acquired immune deficiency syndrome (AIDS), and susceptibility to otherwise innocuous opportunistic infections. However, to establish a successful infection and replicate, the virus has to evade immune control, a task that HIV accomplishes by using a broad array of mechanisms, recently reviewed (Johnson and Desrosiers, 2002). Of particular interest is the inhibition of the CD4⁺ T-cell activity directed to HIV itself (Rosenberg et al, 1997; Norris and Rosenberg, 2001); anti-HIV CD4⁺ T cells are required to establish a CD8⁺ T-cell response capable of controlling the virus (Altfeld and Rosenberg, 2000).

HIV infection of target cells requires fusion of the viral membrane with the cellular membrane; this process is catalyzed by the product of the env gene, the envelope glycoprotein gp160. Mature gp160 is composed of two non-covalently associated subunits—gp120 and gp41 (Wyatt and Sodroski, 1998). Following the interaction of gp120 with membrane receptors on the target cell, the gp41 subunit plays a critical role in virus entry into the target cell. Several functional domains have been identified previously in gp41 (FIG. 1). The N-terminal hydrophobic fusion domain, the fusion peptide (FP), is thought to play a central role in membrane fusion (FIG. 1). Indeed, a mutant FP with a single amino acid (aa) substitution, V2E, shows less fusogenic activity than wild type FP (Kliger et al, 1997). The first 16 aa of FP inserts into the target cell membrane, and the C20 region inserts into the virus membrane (Peisajovich and Shai, 2003; Suarez et al., 2000). The N36 and C34 peptides contain heptad repeats that form a six-helix bundle linker (Chan et al., 1997) that brings the viral and target membranes into close proximity. Fusion can be inhibited by a peptide corresponding to the C terminal heptad repeat, DP178 (amino acids 638-673 of the HIV-1_{LAI} gp41 protein); this peptide is a potent inhibitor of HIV infection, and has been recently approved for human use (Lawless et al., 1996). HIV gp41-derived peptides useful for inhibiting viral infection were disclosed, for example, in U.S. Pat. Nos. 5,464,933, 6,133,418, 6,093,794 (directed to DP178 and to DP107, a peptide corresponding to amino acids 558-595 of the HIV_{LAI} gp41, and analogs thereof) and 5,840,843 (directed to corresponding to amino acid residues 600-862, or a portion thereof comprising the sequence corresponding to amino acid residues 637-666 of the envelope glycoprotein of HIV-1_{IIIB}).

With regard to the interaction of FP with the T-cell membrane, Cladera et al reported that a synthetic peptide encoding the 16 N-terminal aa of FP shows a heterogeneous distribution on the membrane of the Jurkat T-cell line (Cladera et al., 2001). Prominent among the membrane domains of responding T cells is the immune synapse. The immune synapse is the cluster of transmembrane molecules which ensures specific interaction between antigen specific T cells and antigen presenting cells. The immune synapse includes the TCR and the CD4 molecules and other key molecules involved in T-cell activation (Davis and Dustin, 2004; Huppa et al., 2003). Immune synapse function is required for complete T cell activation (Huppa et al., 2003). None of the background art, however, discloses or suggests that the FP domain of HIV-1 gp41 may localize to the immune synapse and regulate T cell activation.

While the normal immune system is closely regulated, aberrations in immune responses are not uncommon. In some instances, the immune system functions inappropriately and reacts to a component of the host as if it were, in fact, foreign. Such a response results in an autoimmune disease, in which the host's immune system attacks the host's own tissue; T cells, as the primary regulators of the immune system, directly or indirectly affect such autoimmune pathologies.

T cell-mediated inflammatory diseases refers to any condition in which an inappropriate T cell response is a component of the disease. This includes both diseases mediated directly by T cells, and also diseases in which an inappropriate T cell response contributes to the production of abnormal antibodies.

Numerous diseases are believed to result from autoimmune mechanisms. Prominent among these are rheumatoid arthritis, systemic lupus erythematosus, multiple sclerosis, Type I diabetes, myasthenia gravis, pemphigus vulgaris. Autoimmune diseases affect millions of individuals worldwide and the cost of these diseases, in terms of actual treatment expenditures and lost productivity, is measured in billions of dollars annually.

T cells also play a major role in the rejection for organ transplantation or graft versus host disease by bone marrow (hematopoietic stem cell) transplantation. Regulation of such immune responses is therefore therapeutically desired.

Traditional reagents and methods used to attempt to regulate an immune response in a patient also result in unwanted side effects and have limited effectiveness. For example, immunosuppressive reagents (e.g., cyclosporin A, azathioprine, and prednisone) used to treat patients with autoimmune diseases also suppress the patient's entire immune response, thereby increasing the risk of infection, and can cause toxic side effects to non-lymphoid tissues. Due to the medical importance of immune regulation and the inadequacies of existing immunopharmacological reagents, reagents and methods to regulate specific parts of the immune system have been the subject of study for many years.

In some autoimmune diseases the relevant autoantigens are known and can therefore be used for specific therapies. For example, methods for inducing immunological tolerance and/or protective immunity to a specific autoantigen have been disclosed for example by WO 01/12222, WO 97/02016 and WO 01/30378 among many others.

Other components of immune responses such as cytokines and adhesion molecules have also been a target for developing immunomodulatory agents, as disclosed, for example by WO 01/57056, U.S. Pat. No. 6,316,420, WO 04/002500 and WO 00/63251 among many others.

Peptides based on TCR derived sequences, for disrupting TCR function presumably by interfering with assembly have also been disclosed (WO 96/22306, WO 97/47644). However, these peptides were demonstrated to be effective at concentrations about 100 fold higher than the peptides of the present invention, thus having a substantially higher potential for toxicity and side effects.

A method of treating or inhibiting symptoms of an autoimmune disease by administering a sub-immunogenic amount of an antigen more immunoreactive with alloimmune-immunogen-absorbed (AIA) serum as compared to nonimmune serum of the same species was disclosed in U.S. Pat. No. 5,230,887. One putative antigen, based on its purported serological cross reactivity with MHC Class II antigens, was suggested to be intact gp41 of HIV. The alleged cross reactivity resides in a C-terminal peptide (Golding et al., 1989).

WO 89/09785 is directed to peptide sequences capable of inhibiting HIV-induced cell fusion or cytopathic syncytia formation, which correspond to a hydrophobic domain located at the amino terminus of gp41 of HIV-1 and the amino terminus of gp40 of HIV-2. The disclosure stipulates that these peptides could have a D-isomer rather than the L-isomer at the amino terminus of the peptide and/or the first two amino acids of the peptide, though no specific embodiment of any peptide comprising a D-amino acid is disclosed. The '785 publication discloses inhibition of HIV-induced fusion and syncytia formation using a family of related peptides, all comprising at least the first three amino acids of gp41 of the BH10 strain; specific peptides correspond to amino acid residues 1-3, a peptide corresponding to amino acid residues 1-6, and a peptide comprising amino acid residues 1-6 in an altered order.

WO 2005/060350 of some of the inventors of the present invention, published after the priority date of the present invention, discloses membrane binding diastereomeric peptides comprising amino acid sequences corresponding to a fragment of a transmembrane protein, wherein at least two amino acid residues of the diastereomeric peptides being in a D-isomer configuration, useful in inhibiting fusion membrane protein events, including specifically viral replication and transmission. The '350 publication discloses, inter alia, the use of diastereomeric peptides corresponding to amino acids 512 to 544 of HIV-1 LAV1 gp41 for inhibiting membrane fusion processes.

There exists a long-felt need for more effective means of curing or ameliorating T cell mediated inflammatory or autoimmune diseases and ameliorating T cell mediated pathologies. The development of new immunosuppressive agents capable of selectively inhibiting the activation of T lymphocytes with minimal side effects is therefore desirable.

### SUMMARY OF THE INVENTION

The present invention discloses for the first time novel uses for peptides derived from the gp41 fusion peptide domain (FP) of HIV or fragments, homologs and derivatives thereof effective in preventing or treating T cell mediated pathologies, including but not limited to inflammatory diseases, autoimmunity and graft rejection. Certain novel active fragments of the gp41 fusion peptide domain (FP) of HIV particularly useful in these methods are claimed as such.

The present invention is based, in part, on the unexpected discovery that the isolated fusion peptide (FP) of the HIV-1 gp41 molecule has therapeutic properties towards T cell mediated inflammatory autoimmune diseases. FP is known in the art to function together with other gp41 domains to mediate virion fusion with host cells. It is now disclosed for the first time that FP co-localizes with the TCR and CD4 molecules in the T cell membrane and is now shown to inhibit T-cell activation in vitro and in vivo. Surprisingly, it was discovered that FP (SEQ ID NO:1) specifically inhibited antigen-specific T-cell proliferation and cytokine secretion while T-cell activation by non specific activators, such as mitogenic antibodies, was not affected. Notably, FP inhibited the activation of arthritogenic T cells and adjuvant arthritis in vivo in animal models of these diseases. In addition, FP was found to be non-immunogenic in vivo, in a sequence and structure dependent manner uncorrelated with its ability to inhibit cell-cell fusion. These unexpected discoveries disclose a novel function of gp41 fusion peptide domain having novel applications for the treatment of T cell mediated pathologies.

Unexpectedly, it was herein discovered that an FP fragment corresponding to amino acid residues 5-13 (SEQ ID NO:407) of SEQ ID NO:1, retain the ability of FP to inhibit antigen-specific T cell proliferation to a greater extent than an PP fragment corresponding to amino acid residues 1-8 (SEQ ID NO:406). The present invention is further based on the unexpected discovery that diastereomeric peptides corresponding to FP or partial sequences thereof inhibit inflammation despite the disruption of the secondary structure of the peptide.

Thus, the present invention provides novel uses for the isolated fusion peptide derived from gp41 of HIV and its fragments, analogs, mutants, variants, conjugates, derivatives and salts, in modulating T cell immunity. The present invention thus relates to the use of both known peptides such as full-length FP (SEQ ID NO:1) its diastereomeric derivative IFFA (SEQ ID NO:6), and the FP mutant V2E (SEQ ID NO:2), as well as peptides not previously described in the art. The invention further provides novel fragments, analogs and variants of FP, as detailed below.

In one aspect, the present invention is directed to the use of an isolated peptide derived from HIV gp41 fusion peptide domain or fragments, analogs, mutants, variants, conjugates, derivatives and salts thereof for the treatment of T cell mediated pathologies, including, but not limited to inflammatory diseases, autoimmune diseases and graft rejection.

According to certain particular embodiments, the disease is a T cell-mediated autoimmune disease including but not limited to: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis), autoimmune hepatitis or rheumatoid arthritis.

The present invention is particularly exemplified herein below by the animal disease model of adjuvant arthritis (AA), a T cell mediated inflammatory autoimmune disease that serves as an experimental model for rheumatoid arthritis. This model is intended as a non-limitative example used for illustrative purposes of the principles of the invention.

In another particular embodiment, the T cell mediated pathology is selected from the group consisting of allograft rejection and graft-versus-host disease.

According to particular embodiments the peptide is a fragment derived from the fusion peptide domain of the gp41 protein of HIV-1. In one preferable embodiment, the peptide has an amino acid sequence as set forth in SEQ ID NO:1 (see Table 1). According to alternative embodiments the peptide is the V2E variant (SEQ ID NO:2; see Table 1). According to certain preferred embodiments, the peptide is an HIV-1 gp41 fusion peptide variant according to Table 2, having an amino acid sequence as set forth in any one of SEQ ID NOS:7-198. According to other preferred embodiments, fusion peptide is an HIV-1 gp41 fusion peptide fragment according to Table 2, having an amino acid sequence as set forth in any one of SEQ ID NOS:199-405. In another particular embodiment, the peptide is an HIV-1 gp41 fusion peptide fragment corresponding to amino acids 1-8 of SEQ ID NO:1, herein designated FP₁₋₈ (SEQ ID NO:406; see Table 1). In another particular embodiment, the peptide is an HIV-1 gp41 fusion peptide fragment corresponding to amino acids 5-13 of SEQ ID NO:1, herein designated FP₅₋₁₃ (SEQ ID NO:407; see Table 1). In another particular embodiment, the fusion peptide is a variant of FP₅₋₁₃ having an amino acid sequence as set forth in any one of SEQ ID NOS:409-414 (see Table 2). It is noted that both shorter active fragments derived from the peptides denoted as SEQ ID NOS:1, 2, 7-407 and 409-414 and longer peptides comprising these sequences are within the scope of the present invention.

In some embodiments, the peptide comprises both D and L amino acids. In another particular embodiment, the peptide is a diastereomeric peptide corresponding to the full length FP, herein designated IFFA (see Table 1) having an amino acid sequence as set forth in SEQ ID NO:6. In another particular embodiment, the peptide is a diastereomeric peptide corresponding to FP₅₋₁₃, herein designated FP_{5-13 A6} (see Table 1) having an amino acid sequence as set forth in SEQ ID NO:408.

In certain embodiment, the peptide includes analogs, variants, derivatives and conjugates of FP₅₋₁₃ capable of inhibiting T cell activation as set forth in formula (I) herein:

X₁-AA₁-AA₂-AA₃-AA₄-AA₅-AA₆-AA₇-AA₈-AA₉-X₂ (1)

wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;
wherein each amino acid can be of either L or D form and the peptide is no more than 30 amino acid residues in length.

In one currently preferred embodiment, the peptide does not contain more than one serine residue.

In another aspect, the invention provides methods for treating or preventing the symptoms of a disease or disorder related to an inappropriate or detrimental T cell response, comprising administering to an individual in need thereof a therapeutically effective amount of a pharmaceutical composition comprising as an active ingredient an isolated peptide derived from HIV gp41 fusion peptide domain or fragments, analogs, variants, conjugates, derivatives and salts thereof.

In one embodiment, the HIV is HIV-1. In another embodiment, the peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids.

In another embodiment, the peptide is a peptide capable of inhibiting T cell activation as set forth in formula (I), as detailed above.

Another aspect of the present invention is a method of inhibiting T-cell activation, wherein said method comprises administering to an individual in need thereof a therapeutically effective amount of a pharmaceutical composition comprising as an active ingredient an isolated peptide derived from HIV gp41 fusion peptide domain or fragments, analogs, variants, conjugates, derivatives and salts thereof.

In one embodiment, the HIV is HIV-1. In another embodiment, the peptide has an amino acid sequence as set forth in any one of SEQ ID NOS:1, 2, and 6-414 or fragments, analogs, variants, derivatives and salts thereof. In another embodiment, the peptide comprises both D and L amino acids.

In another embodiment, the peptide is a peptide capable of inhibiting T cell activation as set forth in formula (I), as detailed above.

Other aspects of the present invention are directed to novel peptides derived from HIV gp41 fusion peptide domain, and pharmaceutical compositions comprising same.

Thus, in another aspect, there is provided a peptide capable of inhibiting T cell activation having the formula (I):

X₁-AA₁-AA₂-AA₃-AA₄-AA₅-AA₆-AA₇-AA₈-AA₉-X₂ (I)

wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;
and wherein each amino acid can be of either L or D form and the peptide is no more than 30 amino acid residues in length.

In one currently preferred embodiment, the peptide does not contain more than one serine residue. In another embodiment, the peptide is FP₅₋₁₃, having an amino acid sequence as set forth in SEQ ID NO:407. In another embodiment, the peptide having an amino acid sequence as set forth in any one of SEQ ID NOS:409-414.

In another embodiment, the peptide comprises both L and D amino acids. In one particular embodiment, the peptide is FP_{5-13 A6}, having an amino acid sequence as set forth in SEQ ID NO:408.

According to one aspect, the present invention provides pharmaceutical compositions comprising as an active ingredient a peptide of formula (I) and salts thereof, and a pharmaceutically acceptable carrier or diluent.

These and other embodiments of the present invention will become apparent in conjunction with the figures, description and claims that follow.

### BRIEF DESCRIPTION OF THE DRAWINGS

FIG. 1 depicts the functional domains of HIV-1 gp41 ectodomain. gp41 becomes active after gp120 binds to surface receptors; FP inserts into the membrane of the target cell; C20 inserts into the membrane of the virion; N36 and C34 form a six-helix bundle “spring” that brings the membranes into apposition; and ISU is immunosuppressive.

FIG. 2 demonstrates the co-localization of FP with the CD4 and TCR molecules in the T-cell immune synapse. FP, V2E or AMP peptides were conjugated to rhodamine (Rho) and used to study peptide binding to the membranes of activated T cells, in combination with FITC-labeled antibodies to CD4 or TCR. (a) Distribution of Rho-labeled FP or AMP in activated T cells. (b) Co-localization of FP-Rho With the CD4 and TCR molecules. (c) Co-localization of V2E-Rho with the TCR. (d) FRET between FP-Rho or V2E-Rho and FITC-labeled antibodies to TCR.

FIG. 3 presents FP inhibition of the T-cell response to Mt. LNC from Mt immunized rats were activated in vitro with the Mt176-90 peptide (a, c and e) or PPD (b, d and f) in the presence of FP (black), V2E (gray) or p277 (white) and the proliferative responses (a and b), IFNγ secretion (c, d) and IL-10 secretion (e, f) were assayed. Similar results were obtained in at least three additional experiments.

FIG. 4 contains a graph showing that FP acts on the T cells and not on the APC in the immune synapse. A2b T cells or APC were separately pre-incubated with FP (black), V2E (gray), or p277 (white) for 2 hours, and washed. The treated T cells were mixed with untreated APC, and the treated APC were mixed with untreated A2b, and the proliferation of the A2b T-cells upon stimulation with Mt176-90 was assayed.

FIG. 5 demonstrates that FP does not inhibit T-cell activation induced by PMA/ionomycin or antibodies to CD3. A2b T cells were stimulated with PMA/ionomycin (a) or antibodies to CD3 (b) in the presence of FP (black) or p277 (white), and T cell proliferation was studied.

FIG. 6 illustrates inhibition of AA by FP. AA was induced by immunization to Mt in oil, mixed with FP (diamonds), V2E (triangles), p277 (hollow squares) or PBS (full squares). Arthritis was scored every two or three days starting at day 10 (a); the leg swelling was measured at day 26 (b); the DTH response to PPD was measured at day 16 (c); and IFNγ secretion was measured at day 26 upon stimulation of LNC with HSP71 or Mt176-90 (d).

FIG. 7 provides a schematic representation of the working hypotheses for FP in HIV infection and in immunotherapy.

FIG. 8 demonstrates FP inhibition of T cell immunity to FP in vivo. LNC from rats immunized to Mt in oil, mixed with FP (triangles), V2E (crosses), IFFA (squares) or PBS (diamonds) were incubated in vitro with FP, V2E, IFFA, or PBS, respectively, and IFNγ secretion was assayed. Similar results were obtained in at least three additional experiments.

FIG. 9 demonstrates that FP-derived fragments and diastereomeric peptides inhibit antigen-specific T cell proliferation. A-C. Proliferative responses of LNC from Mt immunized mice activated in vitro with the MOG₃₅₋₅₅ peptide in the presence or absence of FP₅₋₁₃ (A), FP₁₋₈ (B) or FP_{5-13 A6} (C). D. Proliferative responses of LNC from Mt immunized rats activated in vitro with PPD in the presence or absence of IFFA.

FIG. 10 demonstrates in-vivo inhibition of Adjuvant Arthritis in rats by IFFA. AA was induced by immunization to Mt in oil, mixed with IFFA peptide (circles) or PBS (diamonds). Arthritis was scored every two or three days starting at day 10; standard error was under 10%.

FIG. 11 demonstrates in-vivo inhibition of DTH by dermal treatment with FP and IFFA.

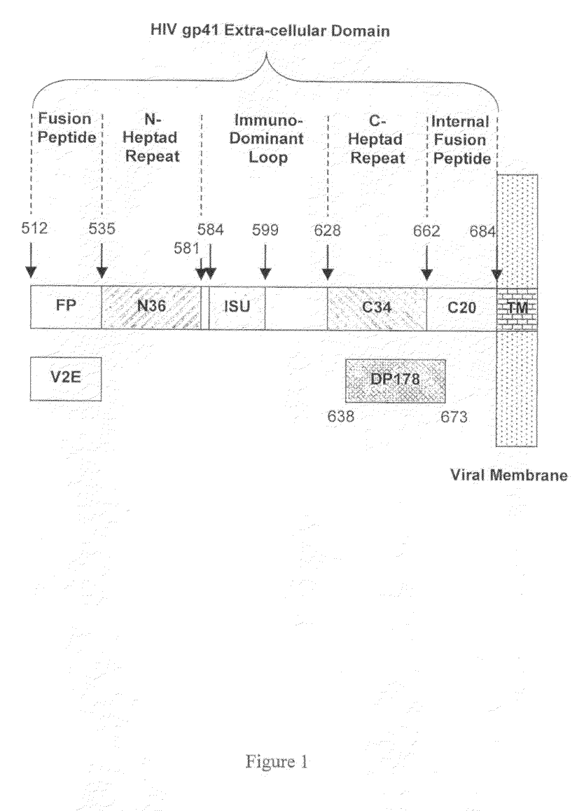

### DETAILED DESCRIPTION OF THE INVENTION

The present invention relates to a novel process for controlling T cell activity. It is now shown for the first time that a composition containing HIV gp41 fusion peptide (FP) is an effective therapeutic reagent for treating T cell-mediated diseases.

Specifically, the present invention relates to the use of the isolated fusion peptide of a human virus in order to inhibit T-cell activation. Preferably the virus is the human immune deficiency virus. More preferably the virus is HIV-1. The isolated fusion peptide is used for preventing or ameliorating T cell-mediated pathologies, such as autoimmune diseases, inflammatory diseases, graft rejection and allergy.

The present invention is based in part on the unexpected discovery that HIV-1 gp41 fusion peptide domain (FP) is able to suppress antigen-specific T cell activation, as will be described in more detail herein.

Without wishing to be bound by a particular mechanism of action it is postulated that the peptides of this invention localize to the immune synapse, thereby inhibiting T cell activation.

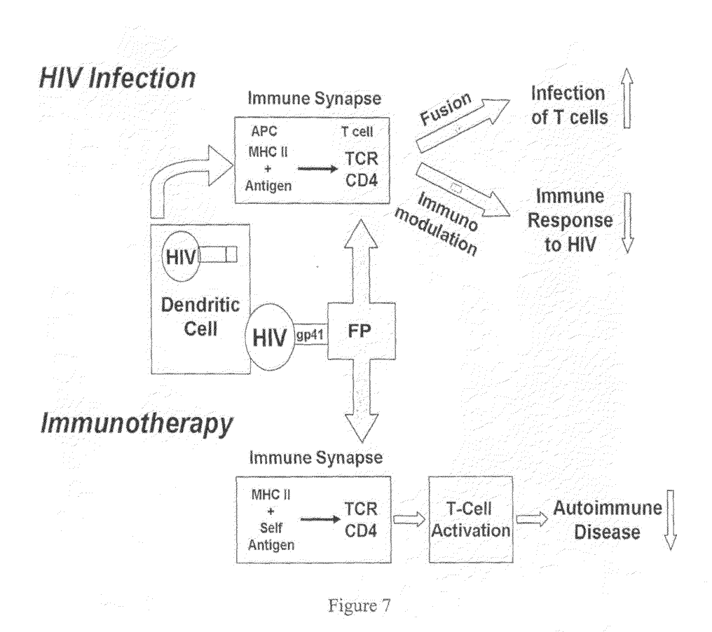

FIG. 7 provides a schematic representation of the postulated functions of FP, whereby FP serves two functions in HIV infection and provides a new tool for immunotherapy. HIV Infection schematically illustrates two effects of FP on HIV infection. Insertion of FP into the T-cell immune synapse facilitates fusion and infection, while it down-regulates specific T-cell immunity to HIV epitopes. Immunotherapy extends the down-regulation by FP to a T-cell mediated immune response under circumstances where the T-cell mediated immune response is deleterious, e.g., a response directed towards self antigens, or graft rejection. Thus, FP itself or an active fragment thereof can serve as a new immunotherapeutic agent.

T Cell Activation

T lymphocytes (T cells) are one of a variety of distinct cell types involved in an immune response. The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell.

Proper activation of T lymphocytes by antigen-presenting cells requires stimulation not only of the TCR, but the combined and coordinated engagement of its co-receptors. Most TCR co-receptors bind cell-surface ligands and are concentrated in areas of cell-cell contact, forming what has been termed an immune synapse. Synapse formation has been associated with the induction of antigen-specific T cell proliferation, cytokine production and lytic granule release, and its function was determined necessary for complete T cell activation (Davis and Dustin, 2004; Huppa et al., 2003).

T Cell Mediated Pathologies

In one aspect, the present invention provides a method for treating a T cell mediated pathology. The term “T-cell mediated pathology” refers to any condition in which an inappropriate or detrimental T cell response is a component of the etiology or pathology of a disease or disorder. The term is intended to include both diseases directly mediated by T cells, and also diseases in which an inappropriate or detrimental T cell response contributes to the production of abnormal antibodies, as well as graft rejection.

In one embodiment of the invention, the composition is useful for treating a T cell-mediated autoimmune disease, including but not limited to: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis) or autoimmune hepatitis, rheumatoid arthritis.

In other embodiments the composition is useful for treating a T cell-mediated inflammatory disease, including but not limited to: inflammatory or allergic diseases such as asthma, hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases); scleroderma; psoriasis (including T-cell mediated psoriasis); dermatitis (including atopic dermatitis and eczematous dermatitis), iritis, conjunctivitis, keratoconjunctivitis, cutaneous lupus erythematosus, scleroderma, vaginitis, proctitis, drug eruptions, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Graves ophthalmopathy and primary biliary cirrhosis.

In other embodiments, the composition is useful for treating graft rejection, including allograft rejection or graft-versus-host disease.

Peptides and Peptide Based Pharmaceutical Compositions

The present invention is based, in part, on the surprising discovery that HIV fusion peptide (FP) and fragments and derivatives thereof inhibit T cell activation. Unexpectedly, it was further discovered, that an isolated FP fragment corresponding to amino acid residues 5-13 of FP, and diastereomeric derivative thereof, and, to a lesser extent, an isolated fragment corresponding to amino acid residues 1-8 of FP, retain the ability of FP to inhibit T cell antigen-dependent proliferation.

Thus, The present invention provides, in a first aspect, a peptide capable of inhibiting T cell activation of the formula (I):

X₁-AA₁-AA₂-AA₃-AA₄-AA₅-AA₆-AA₇-AA₈-AA₉-X₂

wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;

and wherein each amino acid can be of either L or D form and the peptide is no more than 30 amino acid residues in length.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term “hydrophobic amino acid” refers to an amino acid that, on the hydrophobicity scale has a value greater or equal to 0.5, in other words, has a tendency to partition in the nonpolar solvent which is at least equal to that of glycine. Examples of naturally occurring hydrophobic amino acids are aliphatic amino acids alanine, glycine, isoleucine, leucine, methionine, proline, and valine, and aromatic amino acids tryptophan, phenylalanine, and tyrosine. These amino acids confer hydrophobicity as a function of the length of aliphatic and size of aromatic side chains, when found as residues within a protein.

In a currently preferred embodiment, the peptide does not contain more than one serine residue. In one embodiment, the peptide is FP₅₋₁₃, having an amino acid sequence as set forth in SEQ ID NO:407 (GALFLGFLG). Other exemplary embodiments of the generic structure of formula (I) are isolated peptides selected from the group consisting of: GAVFLGFLG (SEQ ID NO:409), GAMFLGFLG (SEQ ID NO:410), GAVLLGFLG (SEQ ID NO:411), GAFFLGFLG (SEQ ID NO:412), GAMIFGFLG (SEQ ID NO:413) and GALLFGFLG (SEQ ID NO:414).

In another embodiment, the peptide is a diastereomeric peptide, i.e. a peptide comprising both L and D amino acids. The diastereomeric peptides may be advantageous over all L- or all D-amino acid peptides having the same amino acid sequence because of their higher water solubility and lower susceptibility to proteolytic degradation. Such characteristics endow the diastereomeric peptides with higher efficacy and higher bioavailability than those of the all L or all D-amino acid peptides comprising the same amino acid sequence. In one particular embodiment, the peptide is FP_{5-13 A6}, having an amino acid sequence as set forth in SEQ ID NO:408 (GALFLGFLG, the D amino acid is bold and underlined).

The peptide of formula (I) is preferably less than 30 amino acids in length. It is to be understood that longer peptides, e.g. up to 50 amino acids in length may also be used for the treatment of T cell mediated pathologies according to the invention. However, shorter peptides are preferable, in one embodiment, for being easier to manufacture. In another currently preferred embodiment, the peptide is no more than 20 amino acids in length. In another currently preferred embodiment, the peptide is no more than 10 amino acids in length. Thus, in certain embodiments, the optional N and C termini, i.e. X₁ and X₂, are up to 20, preferably up to 10, and more preferably 5 or in other embodiments up to 3 amino acids in length or may be absent.

The amino acid sequences of X₁ and X₂ may comprise sequences corresponding to the flanking regions of FP, so long as the peptide is not a peptide previously described in the art. It is noted, that the peptides provided by formula (I) are not intended to include known peptides such as the full length FP (SEQ ID NO:1) and diastereomeric derivatives corresponding to the full length sequence, such as IFFA (SEQ ID NO:6) and other diastereomeric derivatives corresponding to the full length sequence disclosed in WO2005/060350, the known V2E mutant (SEQ ID NO:2) disclosed by Kliger et al. (1997), the peptide corresponding to amino acids 1-16 of SEQ ID NO:1, having an amino acid sequence as set forth in SEQ ID NO:225, disclosed by Martin et al. (1992), and the peptides comprising amino acids 1-3 of SEQ ID NO:1 disclosed by WO 89/09785.

In alternate embodiments, X₁ and X₂ may comprise sequences not derived from FP, so long as they retain the required level of hydrophobicity.

According to another aspect, the present invention is directed to compositions comprising the isolated fusion peptide derived from gp41 of HIV as well as analogs, mutants, variants conjugates, and derivatives thereof.

According to particular embodiments the fusion peptide is derived from the gp41 protein of HIV-1. According to alternative embodiments the fusion peptide is the V2E variant. According to certain preferred embodiments, the fusion peptide has an amino acid sequence as set forth in SEQ ID NO:1. In other preferred embodiments, the fusion peptide has an amino acid sequence as set forth in SEQ ID NO:2. In other preferred embodiments, the fusion peptide is an HIV-1 gp41 fusion peptide variant according to Table 2, having an amino acid sequence as set forth in any one of SEQ ID NOS:7-198. Databases of various HIV strains and variants are available (see, for example, http://www.hiv.lanl.gov/content/index). According to other preferred embodiments, the fusion peptide is an HIV-1 gp41 fusion peptide fragment according to Table 2, having an amino acid sequence as set forth in any one of SEQ ID NOS:199-405. According to other preferred embodiments, the fusion peptide is an HIV-1 gp41 fusion peptide fragment according to Table 2, having an amino acid sequence as set forth in any one of SEQ ID NOS:406-407 and 409-414. In other preferred embodiments, the fusion peptide comprises both L and D amino acid residues. In another preferred embodiment, the fusion peptide has an amino acid sequence as set forth in any one of SEQ ID NOS:6 and 408.

It is noted that both shorter active fragments derived from the peptides denoted as SEQ ID NOS:1, 2 and 6-414 and longer peptides comprising these sequences are within the scope of the present invention. The fusion peptide fragments according to the present invention are preferably 5-50 amino acids in length, more preferably 10-36 amino acids in length.

In other preferred embodiments, the compositions comprise a peptide of formula (I), as detailed above.

The peptides of the invention may be synthesized using the procedures described in detail in the Examples. However, other methods known in the art, including, but not limited to, solid phase (e.g. Boc or f-Moc chemistry) as well as solution phase synthesis methods, may be used for synthesizing the peptides of the invention.

The amino acid residues described herein are preferred to be in the “L” isomeric form. However, residues in the “D” isomeric form can be substituted for any L-amino acid residue, as long as the peptide retains the desired functional property.

It should be understood that a fusion peptide need not be identical to the amino acid sequence of the peptide of the invention, so long as it includes the required sequence and is able to function as the peptide of the invention as described herein.

The present invention encompasses any analog, derivative, and conjugate containing the FP of the invention, so long as the peptide is capable of inhibiting T cell activation. Thus, the present invention encompasses peptides containing non-natural amino acid derivatives or non-protein side chains.

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite inhibitory function on T cells as specified herein.

The term derivative includes any chemical derivative of the peptide of the invention having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a peptide derivative can differ from the natural sequence of the peptides of the invention by chemical modifications including, but are not limited to, terminal-NH₂ acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well known in the art.

Thus, for example, the “blocking groups” represented in formula (I) by X₁ and X₂ are chemical groups that are routinely used in the art of peptide chemistry to confer biochemical stability and resistance to digestion by exopeptidases. Suitable N-terminal protecting groups include, for example, C₁₋₅ alkanoyl groups such as acetyl; other exemplary blocking groups include, without limitation, t-butyloxycarbonyl, methyl, succinyl, methoxysuccinyl, suberyl, adipyl azelayl, dansyl, benzyloxycarbonyl, fluorenylmethoxycarbonyl, methoxyaselayl, methoxyadipyl, methoxysuberyl, and 2,3-dinitrophenyl groups. Also suitable as N-terminal protecting groups are amino acid analogs lacking the amino function. Suitable C-terminal protecting groups include groups which form ketones or amides at the carbon atom of the C-terminal carboxyl, or groups which form esters at the oxygen atom of the carboxyl. Ketone and ester-forming groups include alkyl groups, particularly branched or unbranched C₁₋₅ alkyl groups, e.g., methyl, ethyl, and propyl groups, while amide-forming groups include amino functions such as primary amine, or alkylamino functions, e.g., mono-C₁₋₅ alkylamino and di-C₁₋₅ alkylamino groups such as methylamino, ethylamino, dimethylamino, diethylamino, methylethylamino and the like. Other exemplary blocking groups may include, without limitation, C₃₋₈ cycloalkyl group such as cyclopentyl, cyclohexyl, C₆₋₁₂ aryl group such as phenyl and α-naphthyl, phenyl-C₁₋₂ alkyl group such as benzyl, phenethyl or C7₋₁₄ aralkyl group, C₁₋₂ alkyl group such as α-naphthyl methyl group, and additionally, pivaloyloxymethyl group which is generally used as an oral bioavailable ester. Amino acid analogs are also suitable for protecting the C-terminal end of the present compounds, for example, decarboxylated amino acid analogues such as agmatine.

Peptides of the present invention also include any peptide having one or more additions and/or deletions of residues relative to the sequence of the fusion peptide of the invention, so long as the requisite inhibitory activity is maintained.

Addition of amino acid residues may be performed at either terminus of the peptides of the invention for the purpose of providing a “linker” by which the peptides of this invention can be conveniently bound to a carrier. Such linkers are usually of at least one amino acid residue and can be of 40 or more residues, more often of 1 to 10 residues. Typical amino acid residues used for linking are tyrosine, cysteine, lysine, glutamic and aspartic acid, or the like.

A peptide of the present invention may be coupled to or conjugated with another protein or polypeptide to produce a conjugate. Such a conjugate may have advantages over the peptide used alone. For example, a peptide of the invention may be conjugated to an antigen involved in a T cell mediated pathology. Without wishing to be bound by any theory or mechanism of action, vaccination with such a conjugate may result in reduced T cell activation to the conjugated antigen, and thereby induce a tolerogenic immune response to said disease target antigen. The peptides can be conjugated directly via an amide bond, synthesized as a dual ligand peptide, or joined by means of a linker moiety as is well known in the art to which the present invention pertains.

In another embodiment, the invention relates to a pharmaceutical composition comprising a therapeutically effective amount of an isolated fusion peptide of the invention and a pharmaceutically acceptable carrier.

A pharmaceutical composition useful in the practice of the present invention typically contains a peptide of the invention formulated into the pharmaceutical composition as a neutralized pharmaceutically acceptable salt form. Pharmaceutically acceptable salts include the acid addition salts (formed with the free amino groups of the polypeptide), which are formed with inorganic acids, such as for example, hydrochloric or phosphoric acid, or with organic acids such as acetic, oxalic, tartaric, and the like.

Suitable bases capable of forming salts with the peptides of the present invention include, but are not limited to, inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

The preparation of pharmaceutical compositions, which contain peptides or polypeptides as active ingredients is well known in the art. Typically, such compositions are prepared as indictable, either as liquid solutions or suspensions, however, solid forms, which can be suspended or solubilized prior to injection, can also be prepared. The preparation can also be emulsified. The active therapeutic ingredient is mixed with inorganic and/or organic carriers, which are pharmaceutically acceptable and compatible with the active ingredient. Carriers are pharmaceutically acceptable excipients (vehicles) comprising more or less inert substances when added to a pharmaceutical composition to confer suitable consistency or form to the composition. Suitable carriers are, for example, water, saline, dextrose, glycerol, ethanol, or the like and combinations thereof. In addition, if desired, the composition can contain minor amounts of auxiliary substances such as wetting or emulsifying agents and pH buffering agents, which enhance the effectiveness of the active ingredient.

Therapeutic Use

In one aspect, the present invention provides the use of pharmaceutical compositions comprising the gp41 fusion peptide domain (FP), effective in preventing or treating T Cell mediated pathologies.

One aspect of the present invention is a method of treating an autoimmune disease, wherein said method comprises administering to an individual in need of said treatment a therapeutically effective amount of a pharmaceutical composition comprising an isolated fusion peptide of the invention or an active fragment thereof. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of treating an autoimmune disease; wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

Another aspect of the present invention is a method of preventing the symptoms of an autoimmune disease, wherein said method comprises administering to an individual in need of said treatment a therapeutically effective amount of a pharmaceutical composition comprising an isolated fusion peptide of the invention. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of preventing the symptoms of an autoimmune disease, wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

One aspect of the present invention is a method of treating a T-cell mediated inflammatory disease, wherein said method comprises administering to an individual in need of said treatment a therapeutically effective amount of a pharmaceutical composition comprising an isolated fusion peptide of the invention. In one embodiment, the HIV is HIV-1. In another embodiment, the fusion peptide has an amino acid sequence as set forth in any one of SEQ ID NOS:1, 2, and 6414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of treating a T-cell mediated inflammatory disease, wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

Another aspect of the present invention is a method of preventing the symptoms of a T-cell mediated inflammatory disease, wherein said method comprises administering to an individual in need of said treatment a therapeutically effective amount of a pharmaceutical composition comprising an isolated fusion peptide of the invention. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of preventing the symptoms of a T-cell mediated inflammatory disease, wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

One aspect of the present invention is a method of treating or preventing the symptoms of graft rejection or graft versus host disease, wherein said method comprises administering to an individual in need of said treatment a therapeutically effective amount of a pharmaceutical composition comprising an isolated fusion peptide of the invention. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of a method of treating or preventing the symptoms of graft rejection or graft versus host disease, wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

One aspect of the present invention is a method of inhibiting T-cell activation, wherein said method comprises administering to an individual in need of said treatment a therapeutically effective amount of a pharmaceutical composition comprising an isolated fusion peptide of the invention. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

Another aspect of the present invention is a method of inhibiting T-cell activation, wherein said method comprises administering to an individual in need of said treatment a therapeutically effective amount of a pharmaceutical composition comprising a peptide capable of inhibiting T cell activation according to formula (I) or salts thereof. In another embodiment, the peptide comprises both D and L amino acids.

In another aspect, the invention is directed to the use of a fusion peptide of the invention for the preparation of a pharmaceutical composition for treating or preventing T cell mediated pathologies. In one embodiment, the HIV is HIV-1. In another embodiment, the fusion peptide has an amino acid sequence as set forth in any one of SEQ ID NOS:1, 2, and 6-414 or fragments, analogs, variants, conjugates, derivatives and salts thereof. In another embodiment, the fusion peptide comprises both D and L amino acids. It is noted that both shorter active fragments derived from the peptides denoted as SEQ ID NOS:1, 2 and 6-414 and longer peptides comprising these sequences are within the scope of the present invention.

In another aspect, the invention is directed to the use of a peptide capable of inhibiting T cell activation according to formula (I) for the preparation of a pharmaceutical composition for treating or preventing T cell mediated pathologies.

In another aspect, the pharmaceutical composition can be delivered by a variety of means including intravenous, intramuscularly, infusion, oral, intranasal, intraperitoneal, subcutaneous, rectal, topical, or into other regions, such as into synovial fluids. However delivery of the composition transdermally is also contemplated, such by diffusion via a transdermal patch. For oral ingestion it is possible to prepare peptide analogs or specific peptide formulations having improved oral bioavailability and enhanced resistance to degradation as are known in the art.

The composition is administered in a manner compatible with the dosage formulation, and in a therapeutically effective amount. The quantity to be administered depends on the subject to be treated, capacity of the subject's blood hemostatic system to utilize the active ingredient, and the degree of inhibition of T cell activation or T cell mediated pathology desired. Precise amounts of active ingredient required to be administered depend on the judgment of the practitioner and are peculiar to each individual.

A therapeutically effective amount of a peptide of the invention is an amount that when administered to a patient is capable of inhibiting T cell activation. Assays for detecting the activity of the peptides of the invention may include, but are not limited to, inhibition of T cell antigen-specific proliferation, inhibition of T cell antigen-specific secretion of cytokines such as IFN-γ and IL-10, and inhibition of in vivo disease models including, but not limited to adjuvant arthritis and DTH, as described in the Examples. However, other methods for detecting the inhibition of antigen-specific T cell activation are well known in the art, and may be used for assessing the activity of the peptides of the invention. Preferably, a therapeutically effective amount of a peptide of the present invention is an amount that reduces (inhibits) T cell activation by at least 10 percent, more preferably by at least 50 percent, and most preferably by at least 90 percent, when measured in an in vitro assay or in an in vivo assay. Preferably, a pharmaceutical composition is useful for inhibiting a T cell mediated pathology in a patient as described further herein. In this embodiment, a therapeutically effective amount is an amount that when administered to a patient is sufficient to inhibit, preferably to eradicate, a T cell mediated pathology. A preferred single dose of fusion peptide is from about 5 μg to about 50 mg per kg of body weight, preferably from about 50 μg to about 5 mg per kg of body weight, and more preferably from about 0.125 mg to about 2 mg per kg of body weight. Typically, the physician will determine the actual dosage which will be most suitable for an individual patient and it will vary with the age, weight and response of the particular patient. There can, of course, be individual instances where higher or lower dosage ranges are merited, and such are within the scope of this invention. The fusion peptides of the invention may be administered, for example, as daily or weekly administrations of single doses as described above.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

Peptide Synthesis. The peptides used in this work (presented in Table 1 below) were synthesized using a solid phase method as previously described (Kliger et al., 1997, Merrifield et al., 1982). The synthetic peptides were purified (>98% homogeneity) by reverse-phase HPLC on a C4 column using a linear gradient of 20-60% acetonitrile in 0.05% TFA, for 60 min. The peptides were subjected to amino acid analysis and mass spectrometry to confirm their composition. Unless stated otherwise, stock solutions of concentrated peptides were maintained in DMSO to avoid aggregation of the peptides prior to use. The final concentration of DMSO in each experiment (5% v/v) had no effect on the system under investigation.

TABLE 1
Peptides' designation and sequence.

| | | | | |
|---|---|---|---|---|
| | | | | SEQ |
| | | Start/ | Pro- | ID |
| Designation | Amino Acid Sequence | End | tein | NO: |

| | | | | |
|---|---|---|---|---|
| FP (full | AVGIGALFLGFLGAAGSTM | 512-535 | gp41 | 1 |
| length) | GARSMTLTVQARQL | | | |
| V2E | AEGIGALFLGFLGAAGSTM | 512-535 | gp41 | 2 |
| | GARSMTLTVQARQL | | | |
| AMP | LLKLLKKLLKKLLKL | — | Syn. | 3 |
| p277 | VLGGGCALLRCIPALDSLT | 437-460 | HSP60 | 4 |
| | PANED | | | |
| Mt176-90 | EESNTFGLQLELTEG | 176-190 | HSP65 | 5 |
| IFFA | AVGIGALFLGFLGAAGSTM | — | Syn. | 6 |
| | GARSMTLTVQARQL | | | |
| FP₁₋₈ | AVGIGALF | 512-519 | gp41 | 406 |
| FP₅₋₁₃ | GALFLGFLG | 516-524 | gp41 | 407 |
| FP_{5-13 A6} | GALFLGFLG | — | Syn. | 408 |

Start and End positions are designated according to the HIV-1 HXB2 strain gp160 sequence. The Valine to Glutamic acid mutation at position 2 of the FP is presented in bold face. D-amino acids are presented in bold face and underlined.
gp41, HIV-1 glycoprotein gp41.
AMP, synthetic amphipathic peptide (Papo et al., 2002).
HSP60, human 60 kDa heat shock protein.
HSP65, M tuberculosis 65 kDa heat shock protein.
Syn., synthetic sequence

Fluorescent Labeling of Peptides. The resin-bound peptides were treated with 4-chloro-7-nitrobenz-2-oxa-1,3-diazole fluoride (NBD-F) or 5-carboxytetramethylrhodamine, succinimidyl ester (5-TAMRA, SE (Rhodamine-SE), respectively. The NBD-F and Rhodamine-SE fluorescent probes were purchased from Molecular Probes (City, State, Country). The reaction with NBD-F took place in DMF, and the reaction with Rhodamine in DMF containing 2% diisopropylethylamine as described previously (Gerber and Shai, 2000). The fluorescent probes were used in an excess of 2 equivalents, leading to the formation of resin bound N-terminal NBD or Rhodamine peptides. After 1 h, the resins were washed thoroughly with DMF and then with methylene chloride. The resin was dried under nitrogen flow and then cleaved for 3 hr with TFA 95%, H₂O 2.5% and Triethylsilane 2.5%. The fluorescently-labeled peptides were purified by RP-HPLC (reverse phase high-performance liquid chromatography) on a C4 Bio-Rad semi-preparative column (250×10 mm, 300 Å pore-size, 5-μm particle size) using a gradient of 20%-60% of acetonitrile/water (both containing 0.05% TFA) for 60 min. The purified peptides were shown to be homogeneous (>98%) by analytical RP-HPLC.

Cell lines, antigens and adjuvants. The CD4⁺ T cell clone A2b 21 reacts with the 180-188 epitope of the 65 kDa heat shock protein (HSP65) of M. tuberculosis (Mt), this epitope is contained in the peptide Mt176-90 used herein (van Eden et al., 1988).

Mt Strain H37Ra and incomplete Freund's adjuvant (IFA) were purchased from Difco (Detroit, Mich., USA). Tuberculin purified protein derivative (PPD) was provided by the Statens Serum institute (Copenhagen, Denmark). Purified recombinant 71 kDa heat shock protein (HSP71) was generously provided by Prof. Ruurd van der Zee (Institute of Infectious Diseases and Immunology, Faculty of Veterinary Medicine, Utrecht, The Netherlands). PMA, ionomycin, ovalbumin (OVA) and Concanavalin A (Con A) were purchased from Sigma (Rehovot, Israel).

Co-localization of peptides with membrane molecules. A2b cells were fixed with 4% para-formaldehyde for 15 min on ice and washed with PBS. The cells were then treated with 2% BSA in PBS at room temperature to block unspecific binding. After 30 min the cells were divided into aliquots containing 50,000 cells per 100 μl and either αTCR-FITC or αCD4-FITC were added (1:100) for two hours. The rhodamine-labeled FP or V2E peptides were added during the last 5 min of incubation at a final concentration of 0.5-1 μM. The cells were then washed with PBS and deposited onto a glass slide. The labeled cell samples were observed under a fluorescence confocal microscope. FITC excitation was set at 488 nm, with the laser set at 20% power to minimize bleaching of the fluorophore. Fluorescence was recorded from 505-525 nm. Rhodamine excitation was set at 543 nm, with the laser set at 5% power. Fluorescence data were collected from 560 nm and up.

Fluorescence energy transfer (FRET) between FITC (donor label) and Rhodamine (acceptor label) was detected by the increase in FITC fluorescence in a spot where the Rhodamine probe was bleached. Bleaching was achieved by point excitation at 543 nm for 6 seconds with the laser set to 100%. To verify that the increase in FITC fluorescence was not due to auto-fluorescence, bleaching was performed first using the 488 nm laser and only then at 543 nm. No signal was observed in either 505-525 nm or above 560 nm, eliminating the possibility of auto-fluorescence.

T-cell proliferation. T-cell proliferation assays were performed using either lymph node cells (LNC) or the A2b T cell line, which reacts with the Mt176-90 peptide. Popliteal and inguinal LNC were removed 26 days after the injection of Mt in incomplete Freund's adjuvant (IFA), when strong T cell responses to PPD and Mt176-90 are detectable (Quintana et al., 2002). LNC were cultured at a concentration of 2×10⁵ cells per well; 5×10⁴ A2b T cells were stimulated in the presence of irradiated 5×10⁵ thymic antigen presenting cells (APC) per well, prepared as previously described (van Eden et al., 1985). The cells were plated in quadruplicates in 200 μl round bottom microtiter wells (Costar Corp., Cambridge, USA), with or without antigen, in the presence of various concentrations of the peptides under study. For some experiments, the cells were activated with immobilized anti-CD3 antibodies or PMA/ionomycin as described (Wang et al., 2002). Cultures were incubated for 72 hr at 37° C. in a humidified atmosphere of 7.5% CO₂. T-cell responses were detected by the incorporation of [methyl-3H]-thymidine (Amersham, Buckinghamshire, UK; 1 μCi/well), added during the last 18 hr of incubation. The results of T cell proliferation experiments are shown as the % of inhibition of the T cell proliferation triggered by the antigenin the absence of HIV or control peptides.

Cytokine assays. Supernatants were collected after 72 hr of stimulation, and rat IL-10 and IFNγ were quantified by enzyme-linked immunosorbent assay (ELISA) using Pharmingen's OPTEIA kit (Pharmingen, San Diego, USA) as described (Quintana et al., 2002). When needed, cytokine levels are expressed as percentage of cytokine inhibition relative to cytokine levels when no peptide is present. Otherwise, the cytokines are shown as pg/ml. The lower limits of detection for the experiments described in this paper were 15 pg/ml for IL-10 and IFNγ. Cytokine amounts were calculated based on calibration curves constructed using recombinant cytokines as standards.

Animals. Three-month old female Lewis rats were used in our experiments, raised and maintained under pathogen-free conditions in the Animal Breeding Center of the Weizmann Institute of Science. The experiments were performed under the supervision and guidelines of the Animal Welfare Committee.

Induction and Assessment of Adjuvant Arthritis (AA). To test the effect of FP on T-cell activation in vivo, AA was used as a model system. AA was induced by injecting 50 μl of Mt suspended in IFA (0.5 mg/ml) at the base of the tail. At the time of AA induction, each rat also received 100 μg of FP or control peptide, or PBS dissolved in 50 μl of IFA and mixed with Mt/IFA used to induce AA. The day of AA induction was designated as day 0. Disease severity was assessed by direct observation of all 4 limbs in each animal. A relative score between 0 and 4 was assigned to each limb, based on the degree of joint inflammation, redness and deformity; thus the maximum possible score for an individual animal was 16 (Quintana et al., 2002). The mean AA score (±SEM) is shown for each experimental group. Arthritis was also quantified by measuring hind limb diameter with a caliper. Measurements were taken on the day of the induction of AA and 26 days later (at the peak of AA); the results are presented as the mean±SEM of the difference between the two values for all the animals in each group. The person who scored the disease was blinded to the identity of the groups.

Delayed type hypersensitivity (DTH). Twenty μl of PPD (0.5 mg/ml in PBS) were injected intradermally into the pinna of the right ear on day 16 after AA induction; 20 μl of sterile PBS were injected in the left ear as control. The thickness of the ear was measured 48 hr later using a vernier caliper and expressed as the difference between the right and the left ear.

### Example 1

### FP Co-Localizes with CD4 and TCR in the is

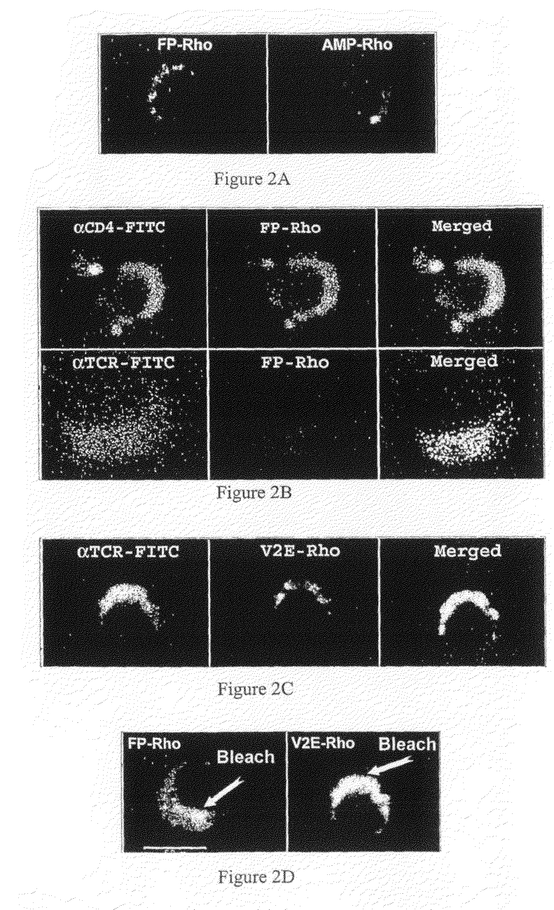

The distribution of FP on the membrane of activated T cells was studied using FP conjugated to Rhodamine (FP-Rho) or NBD (FP-NBD). Rather than uniformly labeling the T-cell membrane, both FP-Rho (FIG. 2A) and FP-NBD inserted themselves into a particular membrane domain. This concentrated distribution contrasted with a control membrane active amphipathic peptide (AMP-Rho) that demonstrated a uniform distribution on the cell membrane (FIG. 2A).

On the membrane of CD4⁺ T cells, the IS domain is marked by the TCR, CD3 and CD4 molecules, among other components (Huppa and Davis, 2003). FIG. 2B shows the localization of the CD4 and TCR molecules at the IS membrane domain. Note that the FP conjugates co-localized with the CD4 and TCR molecules (FIG. 2B). Both FP-Rho (FIG. 2B) and FP-NBD showed the same co-localization; hence distribution to the IS was not limited to a particular fluorophore. In addition, a rhodamine-labeled mutant of FP, V2E (V2E-Rho), showed a similar co-localization with CD4 and the TCR (FIG. 2C).

The co-localization of FP and its V2E mutant was confirmed with the TCR and CD4 receptors by fluorescence energy transfer (FRET) (FIG. 2D). Using a 543 nm laser, a spot on the cell membrane was bleached, thereby reducing the fluorescence of the FP-Rho or V2E-Rho conjugates at that particular spot. A significant increase in the fluorescence of the labeled TCR was observed due to FRET between the labeled, bleached FP (acceptor) and a TCR-specific FITC-conjugated antibody (donor). The average R₀ is under 50 Å for the FITC/Rho pair (Heidecker et al., 1995), therefore, the detection of FRET (FIG. 2D) suggests that the donor and acceptor molecules must be less than 50 Å apart in the membrane. These results suggest that FP binds to the T cell membrane, and preferentially co-localizes closely with the CD4 and TCR molecules at the IS.

### Example 2

### FP Interferes with T-Cell Activation In Vitro

To determine whether the insertion of FP into the IS interferes with T-cell activation, the T-cell response of LNC of Mt immunized rats to the Mt antigen PPD or to the Mt176-90 peptide was studied; these antigens are known to induce strong proliferative responses and cytokine release from T cells in the draining LNC of AA rats (Quintana et al., 2003; Quintana et al., 2002).

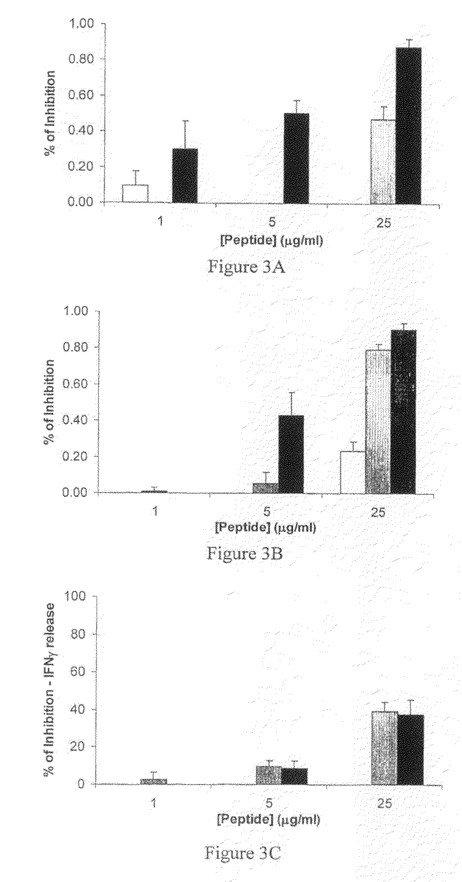

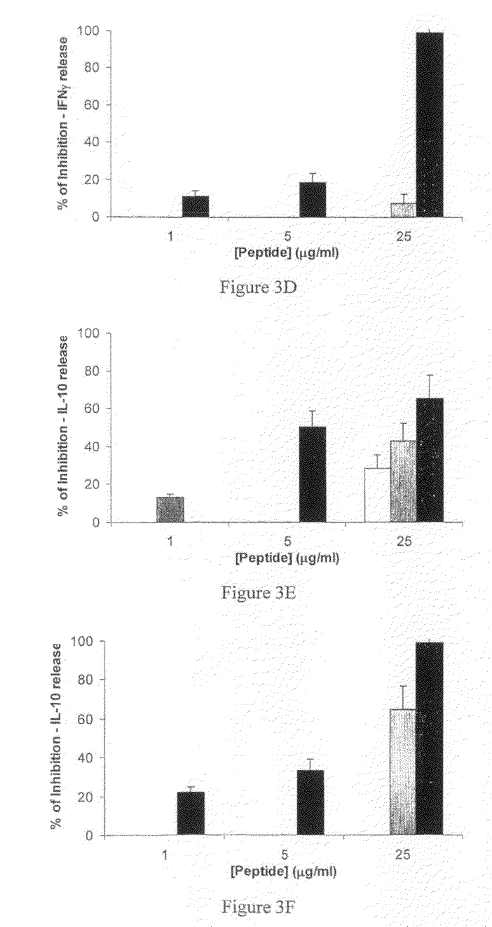

FIGS. 3A and 3B show that FP and the V2E mutant peptide inhibited the T-cell proliferative responses to PPD and to Mt176-90 in a dose-dependent manner. Moreover, the inhibitory effect of V2E was lower than that of FP, suggesting that inhibition is sequence specific and that critical molecular interactions were perturbed by the V to E substitution in V2E (see Table 1). The FP peptide, and to a lesser extent V2E, also inhibited in a dose-dependent manner the secretion of IFNγ and IL-10 triggered by stimulation with PPD or the Mt176-90 peptide (FIGS. 3 C-F). The inhibitory effects of the FP and V2E peptides on antigen-triggered proliferation and cytokine release were not due to cell death, since cells incubated with FP, V2E or the control peptide p277 showed the same survival in culture.

### Example 3

### FP Acts on the T Cells and not on the APC

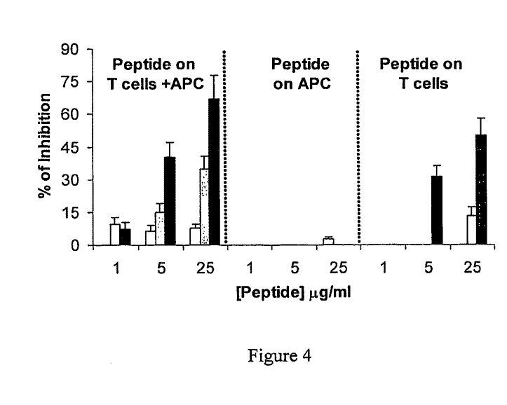

The inhibitory effects of FP on T-cell activation were further studied using a rat CD4⁺ T cell clone, A2b, which proliferates and secretes IFNγ upon stimulation with the Mt176-90 peptide (Quintana et al., 2003; Quintana et al., 2002). Activation of A2b by Mt 176-90 in the presence of FP led to decreased cell proliferation (FIG. 4) and less IFNγ release.

To define the cell targeted by FP in the inhibition of T cell activation, the A2b T cells or the APC were pre-incubated separately with FP, before mixing the cells together with the Mt176-90 peptide. Pre-incubation of the APC had no effect on A2b proliferation (FIG. 4). However, pre-incubation of the A2b T cells with the FP, and not with the control peptide p277, led to a significant inhibition of T cell proliferation (FIG. 4). Thus, FP inhibits T cell activation by directly acting on the T cells rather than on the APC.

### Example 4

### FP does not Inhibit T-Cell Activation Induced by PMA/Ionomycin or Antibodies to CD3

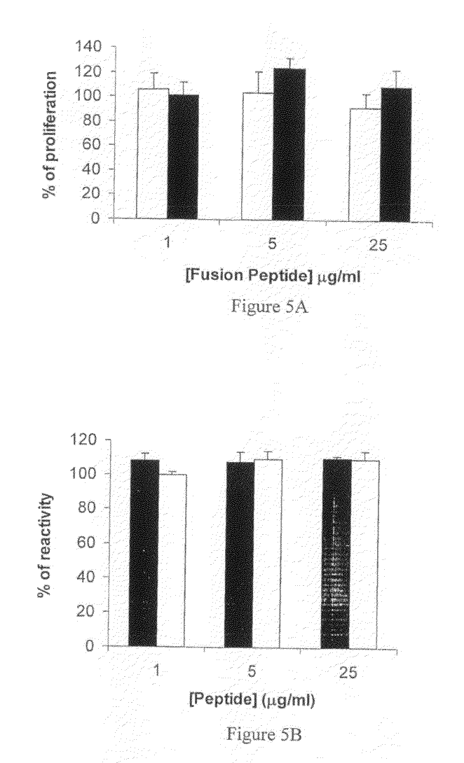

To learn whether FP also can inhibit T-cell activation other than that induced by APC presentation of specific antigen, the effect of FP on T-cell activation induced by PMA/ionomycin (wherein white histograms represent cell receiving 0.5 mg/ml ionomycin and black histograms represent cells receiving 1 mg/ml ionomycin) or a mitogenic monoclonal antibody to CD3 was tested. FP did not inhibit the activation of A2b T cells by either PMA/ionomycin (FIG. 5A) or mitogenic anti-CD3 (FIG. 5B). These findings indicate that FP specifically interferes with T-cell activation induced by the recognition of the MHC-peptide complex presented by the APC.

### Example 5

### FP Inhibits T-Cell Immunity In Vivo

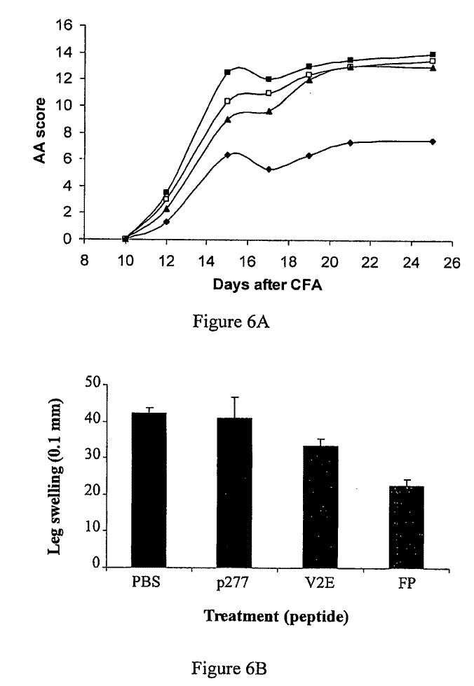

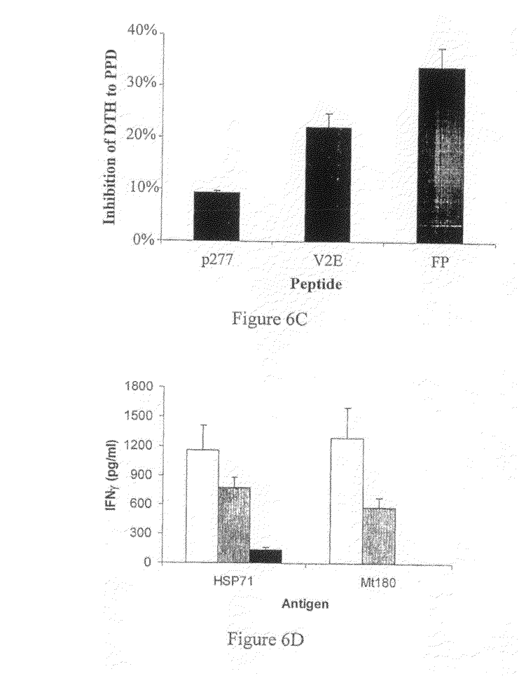

As a test for the inhibitory effects of FP on the activation of specific T cells in vivo, the effects of FP on AA were studied. The immunization of Lewis rats with Mt in oil triggers AA, an experimental autoimmune disease driven by Mt-specific T cells cross-reactive with self-antigens (Holoshitz et al., 1984; Holoshitz et al., 1983). Mt176-90-specific T cells are detectable upon induction of AA (Anderton et al., 1994); indeed the A2b T-cell clone cross-reacts with cartilage and mediates AA (van Eden et al., 1985). Since FP inhibited the T-cell response of primed LNC and of clone A2b to PPD and Mt17-90 in vitro (FIGS. 3 and 4), the effects of FP on the in vivo activation of the T cells that drive AA were investigated. FP administered with the antigen at the time of AA induction led to a significantly milder arthritis, both in terms of clinical score (FIG. 6A) and ankle swelling (FIG. 6B). The control peptides p277 or V2E did not inhibit AA. The mean maximum score was 13.7±0.3 in the control-treated rats, compared to 7.3±0.7 in the FP-treated rats (p<0.05 for both test groups compared to the control group).

The activity of T cells that mediate AA can also be detected in vivo by studying the delayed type hypersensitivity (DTH) response to PPD (van Eden et al., 1985). The DTH response to PPD 16 days after AA induction in rats treated with peptide FP, V2E or p277 was studied. FIG. 6C shows that the administration of FP led to a 35% reduction in the DTH response to PPD, while the inhibition caused by treatment with the V2E or the p277 peptides was 25% and 10%, respectively.

The T cells driving AA manifest a Th1 phenotype; they secrete relatively large amounts of IFNγ upon activation with Mt antigens such as HSP71 or Mt176-90 (Quintana et al., 2003; Quintana et al., 2002). In contrast, the control of AA by various treatments is usually accompanied by a decreased Th1 response (Quintana et al., 2003; Quintana et al., 2002, Tanaka et al., 1999). Note that LNC from FP-treated rats (black histograms) showed reduced secretion of IFNγ upon stimulation with Mycobacterial antigens HSP71 or Mt176-90 (also designated Mt180), while treatment with V2E (gray histograms) or p277 (white histograms) affected IFNγ secretion only slightly (FIG. 6D).

Taken together, these results indicate that the FP can interfere in vivo with T-cell activation induced by specific antigens. This interference led to milder AA (FIGS. 6A and 6B), decreased DTH reactivity (FIG. 6C), and lower IFNγ secretion in response to Mt antigens (FIG. 6D).

### Example 6

### FP Inhibits T Cell Immunity to FP In Vivo

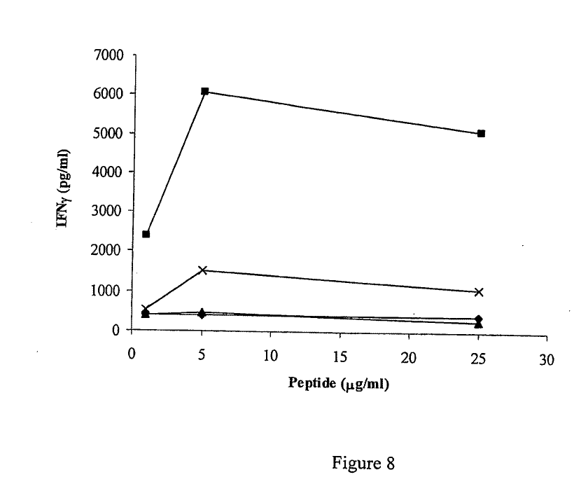

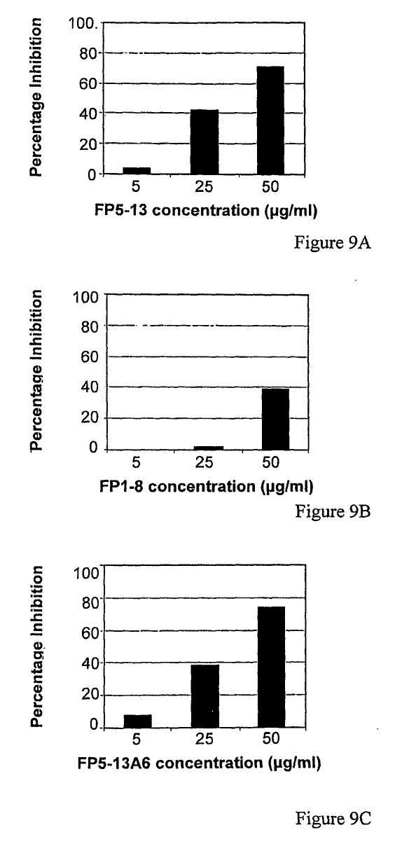

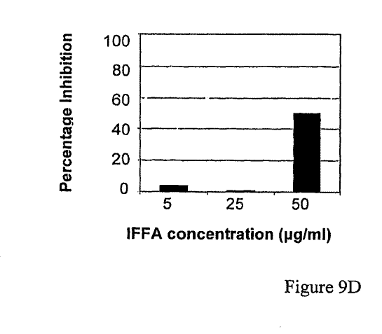

Rats were injected with FP, a control peptide (V2E or IFFA) or PBS in the presence of Mt and IFA. Twenty six days later, LNC were collected and incubated ex vivo with FP, V2E, IFFA, or PBS, respectively, and their IFN-γ secretion level was determined. As can be seen in FIG. 8, LNC from FP injected rats could not be activated by FP ex vivo, as their level of IFN-γ secretion was compatible with that of non-activated. LNC (incubated with PBS). These results indicate low immunogenicity of FP. However, LNC from V2E injected rats showed moderate ex vivo activation with V2E (about five fold higher than that of FP), and LNC from rats injected with the IFFA peptide, a diastereomer derivative of FP in which four D-amino acid residues were incorporated (SEQ ID NO:6), were highly reactive to IFFA ex vivo (about ten fold more than FP). Thus, the immunosuppressive effect of FP is sequence and structure specific. Similar results were obtained when the experiment was repeated with peptides corresponding to the 16 N-terminal amino acids of FP, V2E and IFFA (data not shown).

The incorporation of D amino acids in the IFFA peptide, which was herein shown to substantially increase its immunogenicity, does not affect its ability to inhibit gp41-mediated membrane fusion (Gerber et al., 2004). Hence, the immunosuppressive ability of FP, disclosed herein for the first time, is not merely a reflection its known fusogenic ability, but is rather a newly-identified function residing in the peptide.

### Example 7

### FP-Derived Fragments and Diastereomeric Peptides Interfere with T-Cell Activation In Vitro

The effect of the FP-derived peptides FP₅₋₁₃, FP₁₋₈, FP_{5-13 A6} and IFFA (see Table 1) on antigen-specific T cell proliferation was examined. T-cell proliferation assays were performed using popliteal and inguinal lymph node cells (LNC) removed 26 days after the injection of Mt in incomplete Freund's adjuvant (IFA), as described above. In some experiments, mice LNC were cultured with or without the MOG 35-55 peptide antigen (0.5 μg/ml, FIG. 8A; 0.25 μg/ml, FIGS. 8B-C) in the presence of various concentrations of the peptides under study. In other experiments, rat LNC were cultured with or without PPD antigen (25 μg/ml, FIG. 8D) in the presence of various concentrations of the peptides under study. The results of T cell proliferation experiments are shown as the % of inhibition of the T cell proliferation triggered by the antigen in the absence of HIV or control peptides.

As can be seen in FIG. 8, all the examined peptides inhibited T cell proliferation in a dose-dependant manner, wherein the fragment corresponding to amino acids 5-13 and the diastereomeric peptide thereof inhibited T cell proliferation to a greater extent than the fragment corresponding to amino acids 1-8 of FP

### Example 8

### FP-Derived Diastereomeric Peptide Inhibits T-Cell Immunity In Vivo

To further examine the effect of diastereomeric FP derived peptides on T cell activation in vivo, AA and DTH models were used.

AA was induced in rats as indicated above; at the time of AA induction, each rat also received 100 μg of IFFA or PBS dissolved in 50 μl of IFA and mixed with Mt/IFA used to induce AA. Disease severity was assessed as described above.

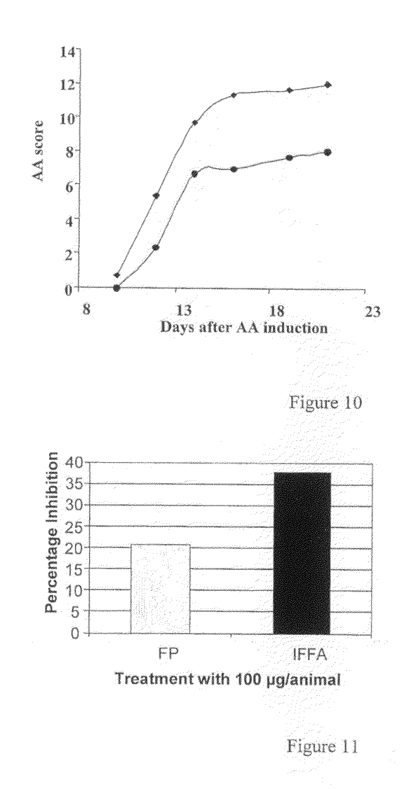

As can be seen in FIG. 10, the administration of IFFA resulted in milder arthritis, determined by AA clinical score.

In other experiments, DTH response to oxazolone in the presence of FP and IFFA was measured. Female Balb/c mice (5 mice per group) were sensitized to the shaved abdominal skin with 100 microliter of 2% oxazolone dissolved in acetone/olive oil (4:1 vol/vol) applied topically, and 5 days later challenged with 20 microliter of 0.5% oxazolone in acetone/olive oil, 10 microliter administered to each side of the ear. One hour after stimulation, FP, IFFA (100 μg in 40 μl DMSO) or DMSO were administered to each side of the ear. A constant area of the ear was measured immediately before challenge and 24 h after challenge.

As can be seen in FIG. 11, both FP and IFFA inhibited the DTH reaction.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

1. Johnson, W. E. & Desrosiers, R. C. Annu. Rev. Med. 53, 499-518 (2002).
2. Rosenberg, E. S. et al. Science 278, 1447-1450 (1997).
3. Norris, P. J. & Rosenberg, E. S. Aids 15 Suppl 2, 16-21 (2001).
4. Aitfeld, M. & Rosenberg, E. S. Curr. Opin. Immunol. 12, 375-380 (2000).
5. Wyatt, R. & Sodroski, J. Science 280, 1884-1888 (1998).
6. Kliger, Y. et al. J. Biol. Chem. 272, 13496-13505 (1997).
7. Peisajovich, S. G. & Shai, Y. Biochim. Biophys. Acta 1614, 122-129 (2003).
8. Suarez, T., Nir, S., Goni, F. M., Saez-Cirion, A. & Nieva, J. L. FEBS Lett 477, 145-149 (2000).
9. Chan, D. C., Fass, D., Berger, J. M. & Kim, P. S. Cell 89, 263-273 (1997).
10. Lawless, M. K. et al. Biochemistry 35, 13697-13708. (1996).
11. Cladera, J., Martin, I. & O'Shea, P. Embo. J. 20, 19-26 (2001).
12. Davis, D. M. & Dustin, M. L. Trends Immunol. 25, 323-327 (2004).
13. Huppa, J. B., Gleimer, M., Sumen, C. & Davis, M. M. Nat. Immunol. 4, 749-755 (2003).
14. Nat. Rev. Immunol. 3, 973-983 (2003).
15. Heidecker, M., Yan-Marriott, Y. & Marriott, G. Biochemistry 34, 11017-11025 (1995).
16. Quintana, F. J., Carmi, P., Mor, F. & Cohen, I. R. J. Immunol. 171, 3533-3541 (2003).
17. Quintana, F. J., Carmi, P., Mor, F. & Cohen, I. R. J. Immunol. 169, 3422-3428 (2002).
18. Holoshitz, J., Matitiau, A. & Cohen, I. R. J. Clin. Invest. 73, 211-215. (1984).
19. Holoshitz, J., Naparstek, Y., Ben-Nun, A. & Cohen, I. R. Science 219, 56-58 (1983).
20. Anderton, S. M., van der Zee, R., Noordzij, A. & van Eden, W. J. Immunol. 152, 3656-3664. (1994).
21. van Eden, W. et al. Proc. Natl. Acad. Sci. USA 82, 5117-5120 (1985).
22. Tanaka, S. et al. J. Immunol. 163, 5560-5565. (1999).
23. Wang, X. M. et al. Clin. Immunol. 105, 199-207 (2002).
24. Merrifield, R. B., Vizioli, L. D. & Boman, H. G. Biochemistry 21, 5020-5031 (1982).
25. Gerber, D. & Shai, Y. J. Biol. Chem. 275, 23602-23607 (2000).
26. van Eden, W. et al. Nature 331, 171-173 (1988).
27. Papo, N., Oren, Z., Pag, U., Sahl, H. G. & Shai, Y. J. Biol. Chem. 277, 33913-33921 (2002).
28. Gerber D, Pritsker M, Gunther-Ausborn S, Johnson B, Blumenthal R, Shai Y. J Biol Chem. 279, 48224-30 (2004).
29. Edelhoch, H. Biochemistry 6, 1948-1954 (1967).
30. Martin et al. Biochem Biophys Acta. 1445, 124-133 (1992).

TABLE 2
fusion peptide variants and active fragments.

| | |
|---|---|
| Amino acid sequence | SEQ ID NO: |

| | |
|---|---|
| AVGIGALFLGFLGAAGSTMGARSMTLTVQARQL | 1 |
| VGIGALFLGFLGAAGSTMGAASMTLTVQARQL | 7 |
| VGLGAVFLGFLGAAGSTMGAASMTLTVQARQL | 8 |
| AAGIGAVLLGFLGAAGSTMGAASMTLTVQARQL | 9 |
| AAGIGAVLPGFLGAAGSTMGAASMTLTVQARQL | 10 |
| AAGIGAVLPGFLGAARSTMGAASMTLTVQARQL | 11 |
| AAGLGAVFLGFLGAAGSTMGAASMTLTVQARQL | 12 |
| AIGIGAMFLGFLGAAGSTMGAASITLTVQARQL | 13 |
| AIGIGAVFIGFLGAAGSTMGAASITLTVQARQL | 14 |
| AIGIGAVFLGFLGAAGSTMGAASITLTVQARQL | 15 |
| AIGIGAVFLGFLGAAGSTMGAASMTLTVQARQL | 16 |
| AIGIGAVFLGFLGTAGSTMGAASITLTVQARQL | 17 |
| AIGIGAVVLGFLGTAGSTMGAASITLTVQARQL | 18 |
| AIGLGAAFLGFLGAAGSTMGAASLTLTVQARQL | 19 |
| AIGLGAAFLGFLGAAGSTMGAASMTLTVQARQL | 20 |
| AIGLGAAFLGFLGAAGSTMGCASMTLTVQARQL | 21 |
| AIGLGAAFLGFLGAAGSTMGVASMTLTVQARQL | 22 |
| AIGLGAALLGFLGAAGSTMGAASMTLTVQARQL | 23 |
| AIGLGALFLGFLGAAGSTMGAASLTLTVQARQL | 24 |
| AIGLGALFLGFLGAAGSTMGAASMTLTVQARQV | 25 |
| AIGLGAMFLGFLGAAGSTMGAASLTLTVQARQL | 26 |
| AIGLGAMFLGFLGAAGSTMGAASMTLTVQARQL | 27 |
| AIGLGAMFLGFLGAAGSTMGAASMTLTVQARQV | 28 |
| AIGLGAMFLGFLGAAGSTMGAASVTLTVQARQL | 29 |
| AIGLGAMFLGFLGAAGSTMGARSLTLTVQARQL | 30 |
| AIGLGAMFLGFLGAAGSTMGARSVTLTVQARQL | 31 |
| AIGLGAMFLGFLGTAGSTMGAASLTLTVQARQL | 32 |
| AIGLGAVFIGFLGAAGSTMGAASITLTVQARQL | 33 |
| AIGLGAVFLGFLGAAGSTMGAASLTLTVQARQL | 34 |
| AIGLGAVFLGFLGAAGSTMGAASMTLTVQARQV | 35 |
| AIGLGAVFXGFLGAAGSTMGAASMTLTVQARQL | 36 |
| AMGIGAVFLGFLGAAGSTMGAASITLTVQARQL | 37 |
| AMGIGAVLLGFLGAPGSTMGAASMTLTVQARQL | 38 |
| AVGIGAALLGFLGAAGSTMGAASMALTVQARQL | 39 |
| AVGIGAFFLGFLGAAGSTMGAASITLTVQARQL | 40 |
| AVGIGAFVLGFLGAAGSTMGAASITLTVQARQL | 41 |
| AVGIGALFLGFLAAAGSTMGAASITLTVQARQL | 42 |
| AVGIGALFLGFLAAAGSTMGARSITLTVQARQL | 43 |
| AVGIGALFLGFLGAAGSAMGAASMTLTVQARQL | 44 |
| AVGIGALFLGFLGAAGSTMGAASITLTVQARQL | 45 |
| AVGIGALFLGFLGAAGSTMGAASLTLTVQARQL | 46 |
| AVGIGALFLGFLGAAGSTMGAASMTLTVQARLL | 47 |
| AVGIGALFLGFLGAAGSTMGAASMTLTVQARQ | 48 |
| AVGIGALFLGFLGAAGSTMGAASMTLTVQARQL | 49 |
| AVGIGALFLGFLGAAGSTMGAASVTLTVQARQL | 50 |
| AVGIGALFLGFLGAAGSTMGCTSMTLTVQARQL | 51 |
| AVGIGALFLGFLGPAGSTMGAASITLTVQARQL | 52 |
| AVGIGALFLGFLGTAGSTMGAASITLTVQARQL | 53 |
| AVGIGALFLGFLGTAGSTMGAASLTLTVQARQL | 54 |
| AVGIGALIFGFLGAAGSTMGAASITLTVQARQL | 55 |
| AVGIGALIFGFLGAAGSTMGAASLTLTVQARQL | 56 |
| AVGIGALLFGFLGAAGSTMGAASMTLTVQARQL | 57 |
| AVGIGALLFGFLGAAGSTMGAASVTLTVQARQL | 58 |
| AVGIGALLFGSLGAAGSTMGAASMTLTVQARLL | 59 |
| AVGIGALLIGFLGAAGSTMGAASMTLTVQARQL | 60 |
| AVGIGALLLGFLGAAGSTMGAASLTLTVQARQL | 61 |
| AVGIGALLLGFLGAAGSTMGAASVTLTVQARQL | 62 |
| AVGIGALLVGFLGAAGSTMGAASMTLTVQARQL | 63 |
| AVGIGALLVGFLGTAGSTMGAASITLTVQARQL | 64 |
| AVGIGALSLGFLGAAGSTMGAASMTLTVQARLL | 65 |
| AVGIGALVFGFLGAAGSTMGAASITLTVQARQL | 66 |
| AVGIGAMFLGFLGAAGSTMGAASITLTVQARQL | 67 |
| AVGIGAMFLGFLGAAGSTMGAASLTLTVQARQL | 68 |
| AVGIGAMFLGFLGAAGSTMGAASMALTVQARQL | 69 |
| AVGIGAMFLGFLGAAGSTMGAASMTLTVQARQL | 70 |
| AVGIGAMFLGFLGAAGSTMGAASVTLTVQARQL | 71 |
| AVGIGAMFLGFLGMAGSTMGAASITLTVQARQL | 72 |
| AVGIGAMFLGILGAAGSTMGAASITLTVQARQL | 73 |
| AVGIGAMFLGILGTAGSAMGAASMTLTVQARQL | 74 |
| AVGIGAMIFGFLGAAGSTMGAASITLTVQARQL | 75 |
| AVGIGAMIFGFLGAAGSTMGAASLTLTVQARQL | 76 |
| AVGIGAMIFGFLGAAGSTMGAASVTLTVQARQL | 77 |
| AVGIGAMIFGFLGAARSTMGAASITLTVQARQL | 78 |
| AVGIGAMIFGFLGAPGSTMGAASITLTVQARQL | 79 |
| AVGIGAMIFGFSGAAGSTMGAASITLTVQARQL | 80 |
| AVGIGAMILGFLGAAGSTMGAASITLTVQARQL | 81 |
| AVGIGAMLFGFLGAAGSTMGAASITLTVQARQL | 82 |
| AVGIGAMLFGFLGAAGSTMGAASMTLTVQARQL | 83 |
| AVGIGAVFFGFLGAAGSTMGAASITLTVQARQL | 84 |
| AVGIGAVFIGFLGAAGSTLGAASMTLTVQARQL | 85 |
| AVGIGAVFIGFLGAAGSTMGAASITLTVQARQL | 86 |
| AVGIGAVFIGFLGAAGSTMGAASMTLTVQARQL | 87 |
| AVGIGAVFIGFLGAAGSTMGAASVTLTVQARQL | 88 |
| AVGIGAVFIGFLSAAGSTMGAASITLTVQARQL | 89 |
| AVGIGAVFLGFLAAAGSSMGAASMTLTVQARQL | 90 |
| AVGIGAVFLGFLAAAGSTMGAASITLTVQARQL | 91 |
| AVGIGAVFLGFLAAAGSTMGAASITLTVQARQV | 92 |
| AVGIGAVFLGFLATAGSTMGAASITLTVQARQL | 93 |
| AVGIGAVFLGFLGAAGSTMGAAAVTLTVQARQL | 94 |
| AVGIGAVFLGFLGAAGSTMGAASITLTIQARQL | 95 |
| AVGIGAVFLGFLGAAGSTMGAASITLTVQARQL | 96 |
| AVGIGAVFLGFLGAAGSTMGAASLTLTVQARQL | 97 |
| AVGIGAVFLGFLGAAGSTMGAASMTLTVQARLL | 98 |
| AVGIGAVFLGFLGAAGSTMGAASMTLTVQARQL | 99 |
| AVGIGAVFLGFLGAAGSTMGAASMTLTVQARRL | 100 |
| AVGIGAVFLGFLGAAGSTMGAASMTVTVQARQL | 101 |
| AVGIGAVFLGFLGAAGSTMGAASTTLTVQARQL | 102 |
| AVGIGAVFLGFLGAAGSTMGAASVTLTVQARQL | 103 |
| AVGIGAVFLGFLGAAGSTMGARSMTLTVQARLL | 104 |
| AVGIGAVFLGFLGAAGSTMGATSITLTVQARQL | 105 |
| AVGIGAVFLGFLGAAGSTMGAVSITLTVQARQL | 106 |
| AVGIGAVFLGFLGAEGSTMGAASLTLTVQARQL | 107 |
| AVGIGAVFLGFLGFAGSTMGAASVTLTVQARQL | 108 |
| AVGIGAVFLGFLGVAGSTMGAASITLTVQARQL | 109 |
| AVGIGAVFLGFLGVAGSTMGAASMTLTVQARQL | 110 |
| AVGIGAVFLGILGAAGSTMGAASITLTVQARQL | 111 |
| AVGIGAVFLGSLGAAGSTMGAASITLTVQARQL | 112 |
| AVGIGAVFLRFLGAAGSTMGAASITLTVQARQL | 113 |
| AVGIGAVFVGFLGAAGSTMGAASITLTVQARQL | 114 |
| AVGIGAVIFGFLGAAGSTMGAASITLTVQARQL | 115 |
| AVGIGAVIFGFLGAAGSTMGAASLTLTVQARQL | 116 |
| AVGIGAVILGFLGAAGSTMGAASITLTVQARQL | 117 |
| AVGIGAVLFGFLGAAGSTMGAASITLTVQARQL | 118 |
| AVGIGAVLFGFLGAAGSTMGAASITLTVQARQV | 119 |
| AVGIGAVLFGFLGAAGSTMGAASLTLTVQARQL | 120 |
| AVGIGAVLIGFLGAAGSTMGAASITLTVQARQL | 121 |
| AVGIGAVLLGFLGAAGSTMGAASITLTVQARQL | 122 |
| AVGIGAVLLGFLGAAGSTMGAASLTLTVQARQL | 123 |
| AVGIGAVLLGFLGAAGSTMGAASMTLTVQARQL | 124 |
| AVGIGAVLLGFLGAAGSTMGAASVTLTVQARQL | 125 |
| AVGIGAVLLGFLGAAGSTMGVASMTLTVQARQL | 126 |
| AVGIGAVLLGFLGTAGSTMGAASITLTVQARQL | 127 |
| AVGIGAVLLGFLGTAGSTMGAASMTLTVQARQL | 128 |
| AVGIGAVLVGFLGAAGSTMGAASITLTVQARQL | 129 |
| AVGIGAVSLGFLGAAGSTMGAASMTLTVQARLL | 130 |
| AVGIGIMIFGFLGAAGSTMGAASITLTVQARQL | 131 |
| AVGIGIMIFGFLGAARSTMGAASITLTVQARQL | 132 |
| AVGIGTMIFGFLGAAGSTMGAASITLTVQARQL | 133 |
| AVGIGVLLLGFLGAAGSTMGAASMALTVQARQL | 134 |
| AVGIGVVFFGFLGAAGSTMGAASITLTVQARQL | 135 |
| AVGLAAVFFGFLGAAGSTMGAASITLTVQARQL | 136 |
| AVGLEAVFLGFLGAAGSTMGAASVTLTVQARQL | 137 |
| AVGLGAAFLGFLGAAGSTMGAASITLTVQARQL | 138 |
| AVGLGAFFLGFLGAAGSTMGAASITLTVQARQL | 139 |
| AVGLGAFFLGFLGVAGSTMGAASITLTVQARQL | 140 |
| AVGLGAIFIGFLGAAGSTMGAASITLTVQARQL | 141 |
| AVGLGALFFGFLGAAGSTMGAASITLTVQARQL | 142 |
| AVGLGALFIGFLGAAGSTMGAAsITLTVQARQL | 143 |
| AVGLGALFIGFLGAAGSTMGAASMTLTVQARQL | 144 |
| AVGLGALFIGFLGAAGSTMGAASVTLTVQARQL | 145 |
| AVGLGALFLGFLGAAGSTMGAASITLTVQARQL | 146 |
| AVGLGALFLGFLGAAGSTMGAASLTLTVQARQL | 147 |
| AVGLGALFLGFLGAAGSTMGAASMTLTVQARQL | 148 |
| AVGLGALFLGFLGAAGSTMGAASVTLTVQARQL | 149 |
| AVGLGALFLGFLGAAGSTMGARSMTLTVQARQL | 150 |
| AVGLGALFLGFLGGAGSTMGAASLTLTVQARQL | 151 |
| AVGLGALLFGFLGAAGSTMGAASITLTVQARQL | 152 |
| AVGLGAMFIGFLGAAGSTMGAASVTLTVQARQL | 153 |
| AVGLGAMFLGFLGAAGSTMGAASITLTVQARQL | 154 |
| AVGLGAMFLGFLGAAGSTMGAASLTLTVQARQL | 155 |
| AVGLGAMFLGFLGAAGSTMGAASMTLTVQARQL | 156 |
| AVGLGAMFLGFLGAAGSTMGAASVTLTVQARQL | 157 |
| AVGLGAMIFGFLGAAGSTMGAASLTLTVQARQL | 158 |
| AVGLGAVFFGFLGAAGSTMGAASITLTVQARQL | 159 |
| AVGLGAVFIGFLGAAGSTMGAASITLTVQARQL | 160 |
| AVGLGAVFIGFLGAAGSTMGAASMTLTVQARQL | 161 |
| AVGLGAVFIGFLGAAGSTMGAASVTLTVQARQL | 162 |
| AVGLGAVFLEFLGAAGSTMGAASVTLTVQARQL | 163 |
| AVGLGAVFLGFLGAAGSTMGAASITLTVQARQL | 164 |
| AVGLGAVFLGFLGAAGSTMGAASLTLTVQARQL | 165 |
| AVGLGAVFLGFLGAAGSTMGAASMTLTVQARQL | 166 |
| AVGLGAVFLGFLGAAGSTMGAASVTLTVQARQL | 167 |
| AVGLGAVFLGFLGAAGSTMGARSITLTVQARQL | 168 |
| AVGLGAVFLGFLGTAGSTMGAASITLTVQARQL | 169 |
| AVGLGAVFLGSLGAAGSTMGAASITLTVQARQL | 170 |
| AVGLGAVFQGFLGAAGSTMGAASITLTVQARQL | 171 |
| AVGLGAVIFGFLGAAGSTMGAASITLTVQARQL | 172 |
| AVGLGAVLFGFLGAAGSTMGAASITLTVQARQL | 173 |
| AVGLGAVLLGFLGAAGSTMGAASITLTVQARQL | 174 |
| AVGLGAVLLGFLGTAGSTMGAASITLTVQARQL | 175 |
| AVGLGVAFLGFLGAAGSTMGAASITLTVQARQL | 176 |
| AVGMAAVFFGFLGAAGSTMGAASITLTVQARQL | 177 |
| AVGMAAVFIGFLGAAGSTMGAASITLTVQARQL | 178 |
| AVGMAAVFLGFLGTAGSTMGAASLTLTVQARQL | 179 |
| AVGMGAFFLGFLGAAGSTMGAASITLTVQARQL | 180 |
| AVGMGAFFLGFLGAAGSTMGAASLTLTVQARQL | 181 |
| AVGMGALFLGFLGAAGSTMGAASITLTVQARQL | 182 |
| AVGMGALFLGFLGAAGSTMGAASLTLTVQARQL | 183 |
| AVGMGALFLGFLGAAGSTMGAASMTLTVQARQL | 184 |
| AVGMGALFLGFLGAAGSTMGAASVTLTVQARQL | 185 |
| AVGMGALFLGFLGAAGSTMGAVSMTLTVQARQL | 186 |
| AVGMGALFLGFLGTAGSTMGAVSMTLTVQARQL | 187 |
| AVGMGALFLGFLSAAGSTMGAASITLTVQARQL | 188 |
| AVGMGAMFLGFLAAAGSTMGAASLTLTVQARQL | 189 |
| AVGMGAMILGFLSAAGSTMGAASITLTVQARQL | 190 |
| AVGMGASFLGFLGAAGSTMGAASITLTVQARQL | 191 |
| AVGMGAVFLGFLGAAGSTMGAASITLTVQARQL | 192 |
| AVGMGAVFLGFLSAAGSTMGAASITLTVQARQL | 193 |
| AVGMGAVLLGFLGAAGSTMGAASITLTVQARQL | 194 |
| AVGVGALFLGFLSAAGSTMGAASITLTVQARQL | 195 |
| AVGVGALLIGFLGAAGSTMGAASMTLTVQARQL | 196 |
| AVGVGAMIFGFLGAAGSTMGAASITLTVQARQL | 197 |
| AVGVGAMILGFLGAAGSTMGAASITLTVQARQL | 198 |
| VGIGALFLGFLGAAG | 199 |
| VGLGAVFLGFLGAAG | 200 |
| AAGIGAVLLGFLGAAG | 201 |
| AAGIGAVLPGFLGAAG | 202 |
| AAGIGAVLPGFLGAAR | 203 |
| AAGLGAVFLGFLGAAG | 204 |
| AIGIGAMFLGFLGAAG | 205 |
| AIGIGAVFIGFLGAAG | 206 |
| AIGIGAVFLGFLGAAG | 207 |
| AIGIGAVFLGFLGTAG | 208 |
| AIGIGAVVLGFLGTAG | 209 |
| AIGLGAAFLGFLGAAG | 210 |
| AIGLGAALLGFLGAAG | 211 |
| AIGLGALFLGFLGAAG | 212 |
| AIGLGAMFLGFLGAAG | 213 |
| AIGLGAMFLGFLGAAG | 214 |
| AIGLGAMFLGFLGTAG | 215 |
| AIGLGAVFIGFLGAAG | 216 |
| AIGLGAVFLGFLGAAG | 217 |
| AIGLGAVFXGFLGAAG | 218 |
| AMGIGAVFLGFLGAAG | 219 |
| AMGIGAVLLGFLGAPG | 220 |
| AVGIGAALLGFLGAAG | 221 |
| AVGIGAFFLGFLGAAG | 222 |
| AVGIGAFVLGFLGAAG | 223 |
| AVGIGALFLGFLAAAG | 224 |
| AVGIGALFLGFLGAAG | 225 |
| AVGIGALFLGFLGPAG | 226 |
| AVGIGALFLGFLGTAG | 227 |
| AVGIGALIFGFLGAAG | 228 |
| AVGIGALLFGFLGAAG | 229 |
| AVGIGALLFGSLGAAG | 230 |
| AVGIGALLIGFLGAAG | 231 |
| AVGIGALLLGFLGAAG | 232 |
| AVGIGALLVGFLGAAG | 233 |
| AVGIGALLVGFLGTAG | 234 |
| AVGIGALSLGFLGAAG | 235 |
| AVGIGALVFGFLGAAG | 236 |
| AVGIGAMFLGFLGAAG | 237 |
| AVGIGAMFLGFLGMAG | 238 |
| AVGIGAMFLGILGAAG | 239 |
| AVGIGAMFLGILGTAG | 240 |
| AVGIGAMIFGFLGAAG | 241 |
| AVGIGAMIFGFLGAAR | 242 |
| AVGIGAMIFGFLGAPG | 243 |
| AVGIGAMIFGFSGAAG | 244 |
| AVGIGAMILGFLGAAG | 245 |
| AVGIGAMLFGFLGAAG | 246 |
| AVGIGAVFFGFLGAAG | 247 |
| AVGIGAVFIGFLGAAG | 248 |
| AVGIGAVFIGFLSAAG | 249 |
| AVGIGAVFLGFLAAAG | 250 |
| AVGIGAVFLGFLATAG | 251 |
| AVGIGAVFLGFLGAAG | 252 |
| AVGIGAVFLGFLGAEG | 253 |
| AVGIGAVFLGFLGFAG | 254 |
| AVGIGAVFLGFLGVAG | 255 |
| AVGIGAVFLGILGAAG | 256 |
| AVGIGAVFLGSLGAAG | 257 |
| AVGIGAVFLRFLGAAG | 258 |
| AVGIGAVFVGFLGAAG | 259 |
| AVGIGAVIFGFLGAAG | 260 |
| AVGIGAVILGFLGAAG | 261 |
| AVGIGAVLFGFLGAAG | 262 |
| AVGIGAVLIGFLGAAG | 263 |
| AVGIGAVLLGFLGAAG | 264 |
| AVGIGAVLLGFLGTAG | 265 |
| AVGIGAVLVGFLGAAG | 266 |
| AVGIGAVSLGFLGAAG | 267 |
| AVGIGIMIFGFLGAAG | 268 |
| AVGIGIMIFGFLGAAR | 269 |
| AVGIGTMIFGFLGAAG | 270 |
| AVGIGVLLLGFLGAAG | 271 |
| AVGIGVVFFGFLGAAG | 272 |
| AVGLAAVFFGFLGAAG | 273 |
| AVGLEAVFLGFLGAAG | 274 |
| AVGLGAAFLGFLGAAG | 275 |
| AVGLGAFFLGFLGAAG | 276 |
| AVGLGAFFLGFLGVAG | 277 |
| AVGLGAIFIGFLGAAG | 278 |
| AVGLGALFFGFLGAAG | 279 |
| AVGLGALFIGFLGAAG | 280 |
| AVGLGALFLGFLGAAG | 281 |
| AVGLGALFLGFLGGAG | 282 |
| AVGLGALLFGFLGAAG | 283 |
| AVGLGAMFIGFLGAAG | 284 |
| AVGLGAMFLGFLGAAG | 285 |
| AVGLGAMIFGFLGAAG | 286 |
| AVGLGAVFFGFLGAAG | 287 |
| AVGLGAVFIGFLGAAG | 288 |
| AVGLGAVFLEFLGAAG | 289 |
| AVGLGAVFLGFLGAAG | 290 |
| AVGLGAVFLGFLGTAG | 291 |
| AVGLGAVFLGSLGAAG | 292 |
| AVGLGAVFQGFLGAAG | 293 |
| AVGLGAVIFGFLGAAG | 294 |
| AVGLGAVLFGFLGAAG | 295 |
| AVGLGAVLLGFLGAAG | 296 |
| AVGLGAVLLGFLGTAG | 297 |
| AVGLGVAFLGFLGAAG | 298 |
| AVGMAAVFFGFLGAAG | 299 |
| AVGMAAVFIGFLGAAG | 300 |
| AVGMAAVFLGFLGTAG | 301 |
| AVGMGAFFLGFLGAAG | 302 |
| AVGMGALFLGFLGAAG | 303 |
| AVGMGALFLGFLGTAG | 304 |
| AVGMGALFLGFLSAAG | 305 |
| AVGMGAMFLGFLAAAG | 306 |
| AVGMGAMILGFLSAAG | 307 |
| AVGMGASFLGFLGAAG | 308 |
| AVGMGAVFLGFLGAAG | 309 |
| AVGMGAVFLGFLSAAG | 310 |
| AVGMGAVLLGFLGAAG | 311 |
| AVGVGALFLGFLSAAG | 312 |
| AVGVGALLIGFLGAAG | 313 |
| AVGVGAMIFGFLGAAG | 314 |
| AVGVGAMILGFLGAAG | 315 |
| VGIGALFLGFL | 316 |
| VGLGAVFLGFL | 317 |
| AAGIGAVLLGFL | 318 |
| AAGIGAVLPGFL | 319 |
| AAGLGAVFLGFL | 320 |
| AIGIGAMFLGFL | 321 |
| AIGIGAVFIGFL | 322 |
| AIGIGAVFLGFL | 323 |
| AIGIGAVVLGFL | 324 |
| AIGLGAAFLGFL | 325 |
| AIGLGAALLGFL | 326 |
| AIGLGALFLGFL | 327 |
| AIGLGAMFLGFL | 328 |
| AIGLGAVFIGFL | 329 |
| AIGLGAVFLGFL | 330 |
| AIGLGAVFXGFL | 331 |
| AIGIGAVFLGFL | 332 |
| AMGIGAVLLGFL | 333 |
| AVGIGAALLGFL | 334 |
| AVGIGAFFLGFL | 335 |
| AVGIGAFVLGFL | 336 |
| AVGIGALFLGFL | 337 |
| AVGIGALIFGFL | 338 |
| AVGIGALLFGFL | 339 |
| AVGIGALLFGSL | 340 |
| AVGIGALLIGFL | 341 |
| AVGIGALLLGFL | 342 |
| AVGIGALLVGFL | 343 |
| AVGIGALSLGFL | 344 |
| AVGIGALVFGFL | 345 |
| AVGIGAMFLGFL | 346 |
| AVGIGAMFLGIL | 347 |
| AVGIGAMIFGFL | 348 |
| AVGIGAMIFGFS | 349 |
| AVGIGAMILGFL | 350 |
| AVGIGAMLFGFL | 351 |
| AVGIGAVFFGFL | 352 |
| AVGIGAVFIGFL | 353 |
| AVGIGAVFLGFL | 354 |
| AVGIGAVFLGIL | 355 |
| AVGIGAVFLGSL | 356 |
| AVGIGAVFLRFL | 357 |
| AVGIGAVGVGFL | 358 |
| AVGIGAVIFGFL | 359 |
| AVGIGAVILGFL | 360 |
| AVGIGAVLFGFL | 361 |
| AVGIGAVLIGFL | 362 |
| AVGIGAVLLGFL | 363 |
| AVGIGAVLVGFL | 364 |
| AVGIGAVSLGFL | 365 |
| AVGIGIMIFGFL | 366 |
| AVGIGTMIFGFL | 367 |
| AVGIGVLLLGFL | 368 |
| AVGIGVVFFGFL | 369 |
| AVGLAAVFFGFL | 370 |
| AVGLEAVFLGFL | 371 |
| AVGLGAAFLGFL | 372 |
| AVGLGAFFLGFL | 373 |
| AVGLGAIFIGFL | 374 |
| AVGLGALFFGFL | 375 |
| AVGLGALFIGFL | 376 |
| AVGLGALFLGFL | 377 |
| AVGLGALLFGFL | 378 |
| AVGLGAMFIGFL | 379 |
| AVGLGAMFLGFL | 380 |
| AVGLGAMIFGFL | 381 |
| AVGLGAVFFGFL | 382 |
| AVGLGAVFIGFL | 383 |
| AVGLGAVFLEFL | 384 |
| AVGLGAVFLGFL | 385 |
| AVGLGAVFLGSL | 386 |
| AVGLGAVFQGFL | 387 |
| AVGLGAVIFGFL | 388 |
| AVGLGAVLFGFL | 389 |
| AVGLGAVLLGFL | 390 |
| AVGLGVAFLGFL | 391 |
| AVGMAAVFFGFL | 392 |
| AVGMAAVFIGFL | 393 |
| AVGMAAVFLGFL | 394 |
| AVGMGAFFLGFL | 395 |
| AVGMGALFLGFL | 396 |
| AVGMGAMFLGFL | 397 |
| AVGMGAMILGFL | 398 |
| AVGMGASFLGFL | 399 |
| AVGMGAVFLGFL | 400 |
| AVGMGAVLLGFL | 401 |
| AVGVGALFLGFL | 402 |
| AVGVGALLIGFL | 403 |
| AVGVGAMIFGFL | 404 |
| AVGVGAMILGFL | 405 |
| AVGIGALF | 406 |
| GALFLGFLG | 407 |
| GAVFLGFLG | 409 |
| GAMFLGFLG | 410 |
| GAVLLGFLG | 411 |
| GAFFLGFLG | 412 |
| GAMIFGFLG | 413 |
| GALLFGFLG | 414 |

1. A method of treating or preventing the symptoms of a disease or disorder related to an inappropriate or detrimental T cell response, comprising administering to an individual in need thereof a therapeutically effective amount of a pharmaceutical composition comprising as an active ingredient an isolated peptide derived from HIV gp41 fusion peptide domain or fragments, analogs, variants, conjugates, derivatives and salts thereof.

2. The method of claim 1, wherein the HIV is HIV-1.

3. The method of claim 1 wherein the fusion peptide has an amino acid sequence as set forth in any one of SEQ ID NOS:1, 2, and 6-414 or fragments, analogs, variants, conjugates, derivatives and salts thereof.

4. The method of claim 1, wherein the fusion peptide has the amino acid sequence AVGIGALFLGFLGAAGSTMGARSMTLTVQARQL (SEQ ID NO:1).

5. The method of claim 1, wherein the fusion peptide has the amino acid sequence AEGIGALFLGFLGAAGSTMGARSMTLTVQARQL (SEQ ID NO:2).

6. The method of claim 1, wherein the fusion peptide has the amino acid sequence AVGIGALFLGFLGAAGSTMGARSMTLTVQARQL, wherein the underlined amino acid residues at positions 3, 6, 9 and 11 are of the “D” isomer configuration (SEQ ID NO:6).

7. The method of claim 1, wherein the fusion peptide has the amino acid sequence AVGIGALF (SEQ ID NO:406).

8. The method of claim 1, wherein the fusion peptide has the amino acid sequence GALFLGFLG (SEQ ID NO:407).

9. The method of claim 1, wherein the fusion peptide has the amino acid sequence GALFLGFLG, wherein the underlined amino acid residue at position 2 is of the “D” isomer configuration (SEQ ID NO:408).

10. The method of claim 1, wherein the fusion peptide is selected from the group consisting of: GAVFLGFLG (SEQ ID NO:409), GAMFLGFLG (SEQ ID NO:410), GAVLLGFLG (SEQ ID NO:411), GAFFLGFLG (SEQ ID NO:412), GAMIFGFLG (SEQ ID NO:413) and GALLFGFLG (SEQ ID NO:414).

11. The method of claim 1 wherein the fusion peptide comprises both D and L amino acids.

12. The method of claim 1 wherein the T cell pathology is an autoimmune disease.

13. The method of claim 12, wherein the autoimmune disease is selected from the group consisting of: multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease (Crohn's and ulcerative colitis), and autoimmune hepatitis.

14. The method of claim 8 wherein the T cell pathology is an inflammatory disease.

15. The method of claim 8 wherein the T cell pathology is selected from graft rejection and graft versus host disease.

16. A method of treating or preventing the symptoms of a disease or disorder related to an inappropriate or detrimental T cell response, comprising administering to an individual in need thereof a therapeutically effective amount of a pharmaceutical composition comprising as an active ingredient a peptide, or salt thereof, capable of inhibiting T cell activation having the formula (I):
X₁-AA₁-AA₂-AA₃-AA₄-AA₅-AA₆-AA₇-AA₈-AA₉-X₂ (I)
wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two-amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;
and wherein each amino acid can be of either L or D form and the peptide is no more than 30 amino acid residues in length.

17. The method of claim 16 wherein the fusion peptide comprises both D and L amino acids.

18. The method of claim 16 wherein said peptide does not contain more than one serine residue.

19. The method of claim 16 wherein the T cell pathology is an autoimmune disease.

20. The method of claim 19, wherein the autoimmune disease is selected from the group consisting of: multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease (Crohn's and ulcerative colitis), and autoimmune hepatitis.

21. The method of claim 16 wherein the T cell pathology is an inflammatory disease.

22. The method of claim 16 wherein the T cell pathology is selected from graft rejection and graft versus host disease.

23. A method of inhibiting T cell activation comprising administering to an individual in need thereof a therapeutically effective amount of a pharmaceutical composition comprising as an active ingredient an isolated peptide derived from HIV gp41 fusion peptide domain or fragments, analogs, variants, conjugates, derivatives and salts thereof.

24. The method of claim 23 wherein the HIV is HIV-1.

25. The method of claim 23 wherein the fusion peptide has an amino acid sequence as set forth in any one of SEQ ID NOS:1, 2, and 6-414 or fragments, analogs, variants, conjugates, derivatives and salts thereof.

26. The method of claim 23 wherein the fusion peptide comprises both D and L amino acids.

27. A method of inhibiting T cell activation comprising administering to an individual in need thereof a therapeutically effective amount of a pharmaceutical composition comprising as an active ingredient a peptide, or salt thereof, capable of inhibiting T cell activation having the formula (I):
X₁-AA₁-AA₂-AA₃-AA₄-AA₅-AA₆-AA₇-AA₈-AA₉-X₂ (I)
wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;
and wherein each amino acid can be of either L or D form and the peptide is no more than 30 amino acid residues in length.

28. The method of claim 27 wherein the fusion peptide comprises both D and L amino acids.

29. The method of claim 27 wherein said peptide does not contain more than one serine residue.

30. A peptide capable of inhibiting T cell activation having the formula (I):
X₁-AA₁-AA₂-AA₃-AA₄-AA₅-AA₆-AA₇-AA₈-AA₉-X₂ (I)
wherein:
X₁ represents an N-terminal blocking group, an amino acid sequence of up to about 20 amino acid residues in length wherein at least 50% of the amino acid residues are hydrophobic and wherein said sequence does not comprise a peptide having the sequence alanine-valine-glycine, or may be absent;
AA₁, AA₂, AA₆ and AA₉ each independently represent an alanine or glycine amino acid residue;
AA₃ represents a phenylalanine, isoleucine, leucine, valine or methionine amino acid residue;
AA₄, AA₅, AA₇ and AA₈ each independently represent a phenylalanine, isoleucine, leucine, valine, methionine or serine amino acid residue, wherein no more than two amino acid residues of AA₄, AA₅, AA₇ and AA₈ are identical, with the exception that any three of AA₄, AA₅, AA₇ and AA₈ may be leucine amino acid residues;
X₂ represents a C-terminal blocking group, an amino acid sequence of up to about 20 amino acid residues in length wherein at least 50% of the amino acid residues are hydrophobic and wherein said sequence does not comprise a peptide having the sequence valine-glutamine-alanine, or may be absent;
and wherein each amino acid can be of either L or D form and the peptide is no more than 30 amino acid residues in length.

31. The peptide of claim 30 wherein said peptide does not contain more than one serine residue.

32. The peptide of claim 30 having the amino acid sequence GALFLGFLG (SEQ ID NO:407).

33. The peptide of claim 30 wherein said peptide is selected from the group consisting of: GAVFLGFLG (SEQ ID NO:409), GAMFLGFLG (SEQ ID NO:410), GAVLLGFLG (SEQ ID NO:411), GAFFLGFLG (SEQ ID NO:412), GAMIFGFLG (SEQ ID NO:413) and GALLFGFLG (SEQ ID NO:414).

34. The peptide of claim 30, wherein said peptide comprises both “L” and “D” amino acids.

35. The peptide of claim 34 having the amino acid sequence GALFLGFLG, wherein the underlined amino acid residue at position 2 is of the “D” isomer configuration (SEQ ID NO:408).

36. A pharmaceutical compositions comprising the peptide of claim 30 and a pharmaceutically acceptable carrier or diluent.


SEQUENCE LISTING (414 sequences)

SEQ ID NO: 1 - protein, 33 aa, Artificial sequence
  Peptides derived of HIV-1 gp41
AVGIGALFLGFLGAAGSTMGARSMTLTVQARQL

SEQ ID NO: 2 - protein, 33 aa, Artificial sequence
  Peptides derived of HIV-1 gp41
AEGIGALFLGFLGAAGSTMGARSMTLTVQARQL

SEQ ID NO: 3 - protein, 15 aa, Artificial sequence
  Synthetic peptide
LLKLLKKLLKKLLKL

SEQ ID NO: 4 - protein, 24 aa, Artificial sequence
  Peptides derived of human 60 kDa heat shock protein
VLGGGCALLRCIPALDSLTPANED

SEQ ID NO: 5 - protein, 15 aa, Artificial sequence
  Peptides derived of M. tuberculosis 65 kDa heat shock protein
EESNTFGLQLELTEG

SEQ ID NO: 6 - protein, 33 aa, Artificial sequence; identical to SEQ ID NO: 1
  Synthetic peptide

SEQ ID NO: 7 - protein, 32 aa, Artificial sequence
  Peptides derived of HIV-1 gp41
VGIGALFLGFLGAAGSTMGAASMTLTVQARQL

SEQ ID NO: 8 - protein, 32 aa, Artificial sequence
  Peptides derived of HIV-1 gp41
VGLGAVFLGFLGAAGSTMGAASMTLTVQARQL

SEQ ID NO: 9 - protein, 33 aa, Artificial sequence
  Peptides derived of HIV-1 gp41
AAGIGAVLLGFLGAAGSTMGAASMTLTVQARQL

SEQ ID NO: 10 - protein, 33 aa, Artificial sequence
  Peptides derived of HIV-1 gp41
AAGIGAVLPGFLGAAGSTMGAASMTLTVQARQL

SEQ ID NO: 11 - protein, 33 aa, Artificial sequence
  Peptides derived of HIV-1 gp41
AAGIGAVLPGFLGAARSTMGAASMTLTVQARQL

SEQ ID NO: 12 - protein, 33 aa, Artificial sequence
  Peptides derived of HIV-1 gp41
AAGLGAVFLGFLGAAGSTMGAASMTLTVQARQL

SEQ ID NO: 13 - protein, 33 aa, Artificial sequence
  Peptides derived of HIV-1 gp41
AIGIGAMFLGFLGAAGSTMGAASITLTVQARQL

SEQ ID NO: 14 - protein, 33 aa, Artificial sequence
  Peptides derived of HIV-1 gp41
AIGIGAVFIGFLGAAGSTMGAASITLTVQARQL

SEQ ID NO: 15 - protein, 33 aa, Artificial sequence
  Peptides derived of HIV-1 gp41
AIGIGAVFLGFLGAAGSTMGAASITLTVQARQL

SEQ ID NO: 16 - protein, 33 aa, Artificial sequence
  Peptides derived of HIV-1 gp41
AIGIGAVFLGFLGAAGSTMGAASMTLTVQARQL

SEQ ID NO: 17 - protein, 33 aa, Artificial sequence
  Peptides derived of HIV-1 gp41
AIGIGAVFLGFLGTAGSTMGAASITLTVQARQL

SEQ ID NO: 18 - protein, 33 aa, Artificial sequence
  Peptides derived of HIV-1 gp41
AIGIGAVVLGFLGTAGSTMGAASITLTVQARQL

SEQ ID NO: 19 - protein, 33 aa, Artificial sequence
  Peptides derived of HIV-1 gp41
AIGLGAAFLGFLGAAGSTMGAASLTLTVQARQL

SEQ ID NO: 20 - protein, 33 aa, Artificial sequence
  Peptides derived of HIV-1 gp41
AIGLGAAFLGFLGAAGSTMGAASMTLTVQARQL

SEQ ID NO: 21 - protein, 33 aa, Artificial sequence
  Peptides derived of HIV-1 gp41
AIGLGAAFLGFLGAAGSTMGCASMTLTVQARQL

SEQ ID NO: 22 - protein, 33 aa, Artificial sequence
  Peptides derived of HIV-1 gp41
AIGLGAAFLGFLGAAGSTMGVASMTLTVQARQL

SEQ ID NO: 23 - protein, 33 aa, Artificial sequence
  Peptides derived of HIV-1 gp41
AIGLGAALLGFLGAAGSTMGAASMTLTVQARQL

SEQ ID NO: 24 - protein, 33 aa, Artificial sequence
  Peptides derived of HIV-1 gp41
AIGLGALFLGFLGAAGSTMGAASLTLTVQARQL

SEQ ID NO: 25 - protein, 33 aa, Artificial sequence
  Peptides derived of HIV-1 gp41
AIGLGALFLGFLGAAGSTMGAASMTLTVQARQV

SEQ ID NO: 26 - protein, 33 aa, Artificial sequence
  Peptides derived of HIV-1 gp41
AIGLGAMFLGFLGAAGSTMGAASLTLTVQARQL

SEQ ID NO: 27 - protein, 33 aa, Artificial sequence
  Peptides derived of HIV-1 gp41
AIGLGAMFLGFLGAAGSTMGAASMTLTVQARQL

SEQ ID NO: 28 - protein, 33 aa, Artificial sequence
  Peptides derived of HIV-1 gp41
AIGLGAMFLGFLGAAGSTMGAASMTLTVQARQV

SEQ ID NO: 29 - protein, 33 aa, Artificial sequence
  Peptides derived of HIV-1 gp41
AIGLGAMFLGFLGAAGSTMGAASVTLTVQARQL

SEQ ID NO: 30 - protein, 33 aa, Artificial sequence
  Peptides derived of HIV-1 gp41
AIGLGAMFLGFLGAAGSTMGARSLTLTVQARQL

SEQ ID NO: 31 - protein, 33 aa, Artificial sequence
  Peptides derived of HIV-1 gp41
AIGLGAMFLGFLGAAGSTMGARSVTLTVQARQL

SEQ ID NO: 32 - protein, 33 aa, Artificial sequence
  Peptides derived of HIV-1 gp41
AIGLGAMFLGFLGTAGSTMGAASLTLTVQARQL

SEQ ID NO: 33 - protein, 33 aa, Artificial sequence
  Peptides derived of HIV-1 gp41
AIGLGAVFIGFLGAAGSTMGAASITLTVQARQL

SEQ ID NO: 34 - protein, 33 aa, Artificial sequence
  Peptides derived of HIV-1 gp41
AIGLGAVFLGFLGAAGSTMGAASLTLTVQARQL

SEQ ID NO: 35 - protein, 33 aa, Artificial sequence
  Peptides derived of HIV-1 gp41
AIGLGAVFLGFLGAAGSTMGAASMTLTVQARQV

SEQ ID NO: 36 - protein, 33 aa, Artificial sequence
  Peptides derived of HIV-1 gp41
AIGLGAVFXGFLGAAGSTMGAASMTLTVQARQL

SEQ ID NO: 37 - protein, 33 aa, Artificial sequence
  Peptides derived of HIV-1 gp41
AMGIGAVFLGFLGAAGSTMGAASITLTVQARQL

SEQ ID NO: 38 - protein, 33 aa, Artificial sequence
  Peptides derived of HIV-1 gp41
AMGIGAVLLGFLGAPGSTMGAASMTLTVQARQL

SEQ ID NO: 39 - protein, 33 aa, Artificial sequence
  Peptides derived of HIV-1 gp41
AVGIGAALLGFLGAAGSTMGAASMALTVQARQL

SEQ ID NO: 40 - protein, 33 aa, Artificial sequence
  Peptides derived of HIV-1 gp41
AVGIGAFFLGFLGAAGSTMGAASITLTVQARQL

SEQ ID NO: 41 - protein, 33 aa, Artificial sequence
  Peptides derived of HIV-1 gp41
AVGIGAFVLGFLGAAGSTMGAASITLTVQARQL

SEQ ID NO: 42 - protein, 33 aa, Artificial sequence
  Peptides derived of HIV-1 gp41
AVGIGALFLGFLAAAGSTMGAASITLTVQARQL

SEQ ID NO: 43 - protein, 33 aa, Artificial sequence
  Peptides derived of HIV-1 gp41
AVGIGALFLGFLAAAGSTMGARSITLTVQARQL

SEQ ID NO: 44 - protein, 33 aa, Artificial sequence
  Peptides derived of HIV-1 gp41
AVGIGALFLGFLGAAGSAMGAASMTLTVQARQL

SEQ ID NO: 45 - protein, 33 aa, Artificial sequence
  Peptides derived of HIV-1 gp41
AVGIGALFLGFLGAAGSTMGAASITLTVQARQL

SEQ ID NO: 46 - protein, 33 aa, Artificial sequence
  Peptides derived of HIV-1 gp41
AVGIGALFLGFLGAAGSTMGAASLTLTVQARQL

SEQ ID NO: 47 - protein, 33 aa, Artificial sequence
  Peptides derived of HIV-1 gp41
AVGIGALFLGFLGAAGSTMGAASMTLTVQARLL

SEQ ID NO: 48 - protein, 32 aa, Artificial sequence
  Peptides derived of HIV-1 gp41
AVGIGALFLGFLGAAGSTMGAASMTLTVQARQ

SEQ ID NO: 49 - protein, 33 aa, Artificial sequence
  Peptides derived of HIV-1 gp41
AVGIGALFLGFLGAAGSTMGAASMTLTVQARQL

SEQ ID NO: 50 - protein, 33 aa, Artificial sequence
  Peptides derived of HIV-1 gp41
AVGIGALFLGFLGAAGSTMGAASVTLTVQARQL

SEQ ID NO: 51 - protein, 33 aa, Artificial sequence
  Peptides derived of HIV-1 gp41
AVGIGALFLGFLGAAGSTMGCTSMTLTVQARQL

SEQ ID NO: 52 - protein, 33 aa, Artificial sequence
  Peptides derived of HIV-1 gp41
AVGIGALFLGFLGPAGSTMGAASITLTVQARQL

SEQ ID NO: 53 - protein, 33 aa, Artificial sequence
  Peptides derived of HIV-1 gp41
AVGIGALFLGFLGTAGSTMGAASITLTVQARQL

SEQ ID NO: 54 - protein, 33 aa, Artificial sequence
  Peptides derived of HIV-1 gp41
AVGIGALFLGFLGTAGSTMGAASLTLTVQARQL

SEQ ID NO: 55 - protein, 33 aa, Artificial sequence
  Peptides derived of HIV-1 gp41
AVGIGALIFGFLGAAGSTMGAASITLTVQARQL

SEQ ID NO: 56 - protein, 33 aa, Artificial sequence
  Peptides derived of HIV-1 gp41
AVGIGALIFGFLGAAGSTMGAASLTLTVQARQL

SEQ ID NO: 57 - protein, 33 aa, Artificial sequence
  Peptides derived of HIV-1 gp41
AVGIGALLFGFLGAAGSTMGAASMTLTVQARQL

SEQ ID NO: 58 - protein, 33 aa, Artificial sequence
  Peptides derived of HIV-1 gp41
AVGIGALLFGFLGAAGSTMGAASVTLTVQARQL

SEQ ID NO: 59 - protein, 33 aa, Artificial sequence
  Peptides derived of HIV-1 gp41
AVGIGALLFGSLGAAGSTMGAASMTLTVQARLL

SEQ ID NO: 60 - protein, 33 aa, Artificial sequence
  Peptides derived of HIV-1 gp41
AVGIGALLIGFLGAAGSTMGAASMTLTVQARQL

SEQ ID NO: 61 - protein, 33 aa, Artificial sequence
  Peptides derived of HIV-1 gp41
AVGIGALLLGFLGAAGSTMGAASLTLTVQARQL

SEQ ID NO: 62 - protein, 33 aa, Artificial sequence
  Peptides derived of HIV-1 gp41
AVGIGALLLGFLGAAGSTMGAASVTLTVQARQL

SEQ ID NO: 63 - protein, 33 aa, Artificial sequence
  Peptides derived of HIV-1 gp41
AVGIGALLVGFLGAAGSTMGAASMTLTVQARQL

SEQ ID NO: 64 - protein, 33 aa, Artificial sequence
  Peptides derived of HIV-1 gp41
AVGIGALLVGFLGTAGSTMGAASITLTVQARQL

SEQ ID NO: 65 - protein, 33 aa, Artificial sequence
  Peptides derived of HIV-1 gp41
AVGIGALSLGFLGAAGSTMGAASMTLTVQARLL

SEQ ID NO: 66 - protein, 33 aa, Artificial sequence
  Peptides derived of HIV-1 gp41
AVGIGALVFGFLGAAGSTMGAASITLTVQARQL

SEQ ID NO: 67 - protein, 33 aa, Artificial sequence
  Peptides derived of HIV-1 gp41
AVGIGAMFLGFLGAAGSTMGAASITLTVQARQL

SEQ ID NO: 68 - protein, 33 aa, Artificial sequence
  Peptides derived of HIV-1 gp41
AVGIGAMFLGFLGAAGSTMGAASLTLTVQARQL

SEQ ID NO: 69 - protein, 33 aa, Artificial sequence
  Peptides derived of HIV-1 gp41
AVGIGAMFLGFLGAAGSTMGAASMALTVQARQL

SEQ ID NO: 70 - protein, 33 aa, Artificial sequence
  Peptides derived of HIV-1 gp41
AVGIGAMFLGFLGAAGSTMGAASMTLTVQARQL

SEQ ID NO: 71 - protein, 33 aa, Artificial sequence
  Peptides derived of HIV-1 gp41
AVGIGAMFLGFLGAAGSTMGAASVTLTVQARQL

SEQ ID NO: 72 - protein, 33 aa, Artificial sequence
  Peptides derived of HIV-1 gp41
AVGIGAMFLGFLGMAGSTMGAASITLTVQARQL

SEQ ID NO: 73 - protein, 33 aa, Artificial sequence
  Peptides derived of HIV-1 gp41
AVGIGAMFLGILGAAGSTMGAASITLTVQARQL

SEQ ID NO: 74 - protein, 33 aa, Artificial sequence
  Peptides derived of HIV-1 gp41
AVGIGAMFLGILGTAGSAMGAASMTLTVQARQL

SEQ ID NO: 75 - protein, 33 aa, Artificial sequence
  Peptides derived of HIV-1 gp41
AVGIGAMIFGFLGAAGSTMGAASITLTVQARQL

SEQ ID NO: 76 - protein, 33 aa, Artificial sequence
  Peptides derived of HIV-1 gp41
AVGIGAMIFGFLGAAGSTMGAASLTLTVQARQL

SEQ ID NO: 77 - protein, 33 aa, Artificial sequence
  Peptides derived of HIV-1 gp41
AVGIGAMIFGFLGAAGSTMGAASVTLTVQARQL

SEQ ID NO: 78 - protein, 33 aa, Artificial sequence
  Peptides derived of HIV-1 gp41
AVGIGAMIFGFLGAARSTMGAASITLTVQARQL

SEQ ID NO: 79 - protein, 33 aa, Artificial sequence
  Peptides derived of HIV-1 gp41
AVGIGAMIFGFLGAPGSTMGAASITLTVQARQL

SEQ ID NO: 80 - protein, 33 aa, Artificial sequence
  Peptides derived of HIV-1 gp41
AVGIGAMIFGFSGAAGSTMGAASITLTVQARQL

SEQ ID NO: 81 - protein, 33 aa, Artificial sequence
  Peptides derived of HIV-1 gp41
AVGIGAMILGFLGAAGSTMGAASITLTVQARQL

SEQ ID NO: 82 - protein, 33 aa, Artificial sequence
  Peptides derived of HIV-1 gp41
AVGIGAMLFGFLGAAGSTMGAASITLTVQARQL

SEQ ID NO: 83 - protein, 33 aa, Artificial sequence
  Peptides derived of HIV-1 gp41
AVGIGAMLFGFLGAAGSTMGAASMTLTVQARQL

SEQ ID NO: 84 - protein, 33 aa, Artificial sequence
  Peptides derived of HIV-1 gp41
AVGIGAVFFGFLGAAGSTMGAASITLTVQARQL

SEQ ID NO: 85 - protein, 33 aa, Artificial sequence
  Peptides derived of HIV-1 gp41
AVGIGAVFIGFLGAAGSTLGAASMTLTVQARQL

SEQ ID NO: 86 - protein, 33 aa, Artificial sequence
  Peptides derived of HIV-1 gp41
AVGIGAVFIGFLGAAGSTMGAASITLTVQARQL

SEQ ID NO: 87 - protein, 33 aa, Artificial sequence
  Peptides derived of HIV-1 gp41
AVGIGAVFIGFLGAAGSTMGAASMTLTVQARQL

SEQ ID NO: 88 - protein, 33 aa, Artificial sequence
  Peptides derived of HIV-1 gp41
AVGIGAVFIGFLGAAGSTMGAASVTLTVQARQL

SEQ ID NO: 89 - protein, 33 aa, Artificial sequence
  Peptides derived of HIV-1 gp41
AVGIGAVFIGFLSAAGSTMGAASITLTVQARQL

SEQ ID NO: 90 - protein, 33 aa, Artificial sequence
  Peptides derived of HIV-1 gp41
AVGIGAVFLGFLAAAGSSMGAASMTLTVQARQL

SEQ ID NO: 91 - protein, 33 aa, Artificial sequence
  Peptides derived of HIV-1 gp41
AVGIGAVFLGFLAAAGSTMGAASITLTVQARQL

SEQ ID NO: 92 - protein, 33 aa, Artificial sequence
  Peptides derived of HIV-1 gp41
AVGIGAVFLGFLAAAGSTMGAASITLTVQARQV

SEQ ID NO: 93 - protein, 33 aa, Artificial sequence
  Peptides derived of HIV-1 gp41
AVGIGAVFLGFLATAGSTMGAASITLTVQARQL

SEQ ID NO: 94 - protein, 33 aa, Artificial sequence
  Peptides derived of HIV-1 gp41
AVGIGAVFLGFLGAAGSTMGAAAVTLTVQARQL

SEQ ID NO: 95 - protein, 33 aa, Artificial sequence
  Peptides derived of HIV-1 gp41
AVGIGAVFLGFLGAAGSTMGAASITLTIQARQL

SEQ ID NO: 96 - protein, 33 aa, Artificial sequence
  Peptides derived of HIV-1 gp41
AVGIGAVFLGFLGAAGSTMGAASITLTVQARQL

SEQ ID NO: 97 - protein, 33 aa, Artificial sequence
  Peptides derived of HIV-1 gp41
AVGIGAVFLGFLGAAGSTMGAASLTLTVQARQL

SEQ ID NO: 98 - protein, 33 aa, Artificial sequence
  Peptides derived of HIV-1 gp41
AVGIGAVFLGFLGAAGSTMGAASMTLTVQARLL

SEQ ID NO: 99 - protein, 33 aa, Artificial sequence
  Peptides derived of HIV-1 gp41
AVGIGAVFLGFLGAAGSTMGAASMTLTVQARQL

SEQ ID NO: 100 - protein, 33 aa, Artificial sequence
  Peptides derived of HIV-1 gp41
AVGIGAVFLGFLGAAGSTMGAASMTLTVQARRL

SEQ ID NO: 101 - protein, 33 aa, Artificial sequence
  Peptides derived of HIV-1 gp41
AVGIGAVFLGFLGAAGSTMGAASMTVTVQARQL

SEQ ID NO: 102 - protein, 33 aa, Artificial sequence
  Peptides derived of HIV-1 gp41
AVGIGAVFLGFLGAAGSTMGAASTTLTVQARQL

SEQ ID NO: 103 - protein, 33 aa, Artificial sequence
  Peptides derived of HIV-1 gp41
AVGIGAVFLGFLGAAGSTMGAASVTLTVQARQL

SEQ ID NO: 104 - protein, 33 aa, Artificial sequence
  Peptides derived of HIV-1 gp41
AVGIGAVFLGFLGAAGSTMGARSMTLTVQARLL

SEQ ID NO: 105 - protein, 33 aa, Artificial sequence
  Peptides derived of HIV-1 gp41
AVGIGAVFLGFLGAAGSTMGATSITLTVQARQL

SEQ ID NO: 106 - protein, 33 aa, Artificial sequence
  Peptides derived of HIV-1 gp41
AVGIGAVFLGFLGAAGSTMGAVSITLTVQARQL

SEQ ID NO: 107 - protein, 33 aa, Artificial sequence
  Peptides derived of HIV-1 gp41
AVGIGAVFLGFLGAEGSTMGAASLTLTVQARQL

SEQ ID NO: 108 - protein, 33 aa, Artificial sequence
  Peptides derived of HIV-1 gp41
AVGIGAVFLGFLGFAGSTMGAASVTLTVQARQL

SEQ ID NO: 109 - protein, 33 aa, Artificial sequence
  Peptides derived of HIV-1 gp41
AVGIGAVFLGFLGVAGSTMGAASITLTVQARQL

SEQ ID NO: 110 - protein, 33 aa, Artificial sequence
  Peptides derived of HIV-1 gp41
AVGIGAVFLGFLGVAGSTMGAASMTLTVQARQL

SEQ ID NO: 111 - protein, 33 aa, Artificial sequence
  Peptides derived of HIV-1 gp41
AVGIGAVFLGILGAAGSTMGAASITLTVQARQL

SEQ ID NO: 112 - protein, 33 aa, Artificial sequence
  Peptides derived of HIV-1 gp41
AVGIGAVFLGSLGAAGSTMGAASITLTVQARQL

SEQ ID NO: 113 - protein, 33 aa, Artificial sequence
  Peptides derived of HIV-1 gp41
AVGIGAVFLRFLGAAGSTMGAASITLTVQARQL

SEQ ID NO: 114 - protein, 33 aa, Artificial sequence
  Peptides derived of HIV-1 gp41
AVGIGAVFVGFLGAAGSTMGAASITLTVQARQL

SEQ ID NO: 115 - protein, 33 aa, Artificial sequence
  Peptides derived of HIV-1 gp41
AVGIGAVIFGFLGAAGSTMGAASITLTVQARQL

SEQ ID NO: 116 - protein, 33 aa, Artificial sequence
  Peptides derived of HIV-1 gp41
AVGIGAVIFGFLGAAGSTMGAASLTLTVQARQL

SEQ ID NO: 117 - protein, 33 aa, Artificial sequence
  Peptides derived of HIV-1 gp41
AVGIGAVILGFLGAAGSTMGAASITLTVQARQL

SEQ ID NO: 118 - protein, 33 aa, Artificial sequence
  Peptides derived of HIV-1 gp41
AVGIGAVLFGFLGAAGSTMGAASITLTVQARQL

SEQ ID NO: 119 - protein, 33 aa, Artificial sequence
  Peptides derived of HIV-1 gp41
AVGIGAVLFGFLGAAGSTMGAASITLTVQARQV

SEQ ID NO: 120 - protein, 33 aa, Artificial sequence
  Peptides derived of HIV-1 gp41
AVGIGAVLFGFLGAAGSTMGAASLTLTVQARQL

SEQ ID NO: 121 - protein, 33 aa, Artificial sequence
  Peptides derived of HIV-1 gp41
AVGIGAVLIGFLGAAGSTMGAASITLTVQARQL

SEQ ID NO: 122 - protein, 33 aa, Artificial sequence
  Peptides derived of HIV-1 gp41
AVGIGAVLLGFLGAAGSTMGAASITLTVQARQL

SEQ ID NO: 123 - protein, 33 aa, Artificial sequence
  Peptides derived of HIV-1 gp41
AVGIGAVLLGFLGAAGSTMGAASLTLTVQARQL

SEQ ID NO: 124 - protein, 33 aa, Artificial sequence
  Peptides derived of HIV-1 gp41
AVGIGAVLLGFLGAAGSTMGAASMTLTVQARQL

SEQ ID NO: 125 - protein, 33 aa, Artificial sequence
  Peptides derived of HIV-1 gp41
AVGIGAVLLGFLGAAGSTMGAASVTLTVQARQL

SEQ ID NO: 126 - protein, 33 aa, Artificial sequence
  Peptides derived of HIV-1 gp41
AVGIGAVLLGFLGAAGSTMGVASMTLTVQARQL

SEQ ID NO: 127 - protein, 33 aa, Artificial sequence
  Peptides derived of HIV-1 gp41
AVGIGAVLLGFLGTAGSTMGAASITLTVQARQL

SEQ ID NO: 128 - protein, 33 aa, Artificial sequence
  Peptides derived of HIV-1 gp41
AVGIGAVLLGFLGTAGSTMGAASMTLTVQARQL

SEQ ID NO: 129 - protein, 33 aa, Artificial sequence
  Peptides derived of HIV-1 gp41
AVGIGAVLVGFLGAAGSTMGAASITLTVQARQL

SEQ ID NO: 130 - protein, 33 aa, Artificial sequence
  Peptides derived of HIV-1 gp41
AVGIGAVSLGFLGAAGSTMGAASMTLTVQARLL

SEQ ID NO: 131 - protein, 33 aa, Artificial sequence
  Peptides derived of HIV-1 gp41
AVGIGIMIFGFLGAAGSTMGAASITLTVQARQL

SEQ ID NO: 132 - protein, 33 aa, Artificial sequence
  Peptides derived of HIV-1 gp41
AVGIGIMIFGFLGAARSTMGAASITLTVQARQL

SEQ ID NO: 133 - protein, 33 aa, Artificial sequence
  Peptides derived of HIV-1 gp41
AVGIGTMIFGFLGAAGSTMGAASITLTVQARQL

SEQ ID NO: 134 - protein, 33 aa, Artificial sequence
  Peptides derived of HIV-1 gp41
AVGIGVLLLGFLGAAGSTMGAASMALTVQARQL

SEQ ID NO: 135 - protein, 33 aa, Artificial sequence
  Peptides derived of HIV-1 gp41
AVGIGVVFFGFLGAAGSTMGAASITLTVQARQL

SEQ ID NO: 136 - protein, 33 aa, Artificial sequence
  Peptides derived of HIV-1 gp41
AVGLAAVFFGFLGAAGSTMGAASITLTVQARQL

SEQ ID NO: 137 - protein, 33 aa, Artificial sequence
  Peptides derived of HIV-1 gp41
AVGLEAVFLGFLGAAGSTMGAASVTLTVQARQL

SEQ ID NO: 138 - protein, 33 aa, Artificial sequence
  Peptides derived of HIV-1 gp41
AVGLGAAFLGFLGAAGSTMGAASITLTVQARQL

SEQ ID NO: 139 - protein, 33 aa, Artificial sequence
  Peptides derived of HIV-1 gp41
AVGLGAFFLGFLGAAGSTMGAASITLTVQARQL

SEQ ID NO: 140 - protein, 33 aa, Artificial sequence
  Peptides derived of HIV-1 gp41
AVGLGAFFLGFLGVAGSTMGAASITLTVQARQL

SEQ ID NO: 141 - protein, 33 aa, Artificial sequence
  Peptides derived of HIV-1 gp41
AVGLGAIFIGFLGAAGSTMGAASITLTVQARQL

SEQ ID NO: 142 - protein, 33 aa, Artificial sequence
  Peptides derived of HIV-1 gp41
AVGLGALFFGFLGAAGSTMGAASITLTVQARQL

SEQ ID NO: 143 - protein, 33 aa, Artificial sequence
  Peptides derived of HIV-1 gp41
AVGLGALFIGFLGAAGSTMGAASITLTVQARQL

SEQ ID NO: 144 - protein, 33 aa, Artificial sequence
  Peptides derived of HIV-1 gp41
AVGLGALFIGFLGAAGSTMGAASMTLTVQARQL

SEQ ID NO: 145 - protein, 33 aa, Artificial sequence
  Peptides derived of HIV-1 gp41
AVGLGALFIGFLGAAGSTMGAASVTLTVQARQL

SEQ ID NO: 146 - protein, 33 aa, Artificial sequence
  Peptides derived of HIV-1 gp41
AVGLGALFLGFLGAAGSTMGAASITLTVQARQL

SEQ ID NO: 147 - protein, 33 aa, Artificial sequence
  Peptides derived of HIV-1 gp41
AVGLGALFLGFLGAAGSTMGAASLTLTVQARQL

SEQ ID NO: 148 - protein, 33 aa, Artificial sequence
  Peptides derived of HIV-1 gp41
AVGLGALFLGFLGAAGSTMGAASMTLTVQARQL

SEQ ID NO: 149 - protein, 33 aa, Artificial sequence
  Peptides derived of HIV-1 gp41
AVGLGALFLGFLGAAGSTMGAASVTLTVQARQL

SEQ ID NO: 150 - protein, 33 aa, Artificial sequence
  Peptides derived of HIV-1 gp41
AVGLGALFLGFLGAAGSTMGARSMTLTVQARQL

SEQ ID NO: 151 - protein, 33 aa, Artificial sequence
  Peptides derived of HIV-1 gp41
AVGLGALFLGFLGGAGSTMGAASLTLTVQARQL

SEQ ID NO: 152 - protein, 33 aa, Artificial sequence
  Peptides derived of HIV-1 gp41
AVGLGALLFGFLGAAGSTMGAASITLTVQARQL

SEQ ID NO: 153 - protein, 33 aa, Artificial sequence
  Peptides derived of HIV-1 gp41
AVGLGAMFIGFLGAAGSTMGAASVTLTVQARQL

SEQ ID NO: 154 - protein, 33 aa, Artificial sequence
  Peptides derived of HIV-1 gp41
AVGLGAMFLGFLGAAGSTMGAASITLTVQARQL

SEQ ID NO: 155 - protein, 33 aa, Artificial sequence
  Peptides derived of HIV-1 gp41
AVGLGAMFLGFLGAAGSTMGAASLTLTVQARQL

SEQ ID NO: 156 - protein, 33 aa, Artificial sequence
  Peptides derived of HIV-1 gp41
AVGLGAMFLGFLGAAGSTMGAASMTLTVQARQL

SEQ ID NO: 157 - protein, 33 aa, Artificial sequence
  Peptides derived of HIV-1 gp41
AVGLGAMFLGFLGAAGSTMGAASVTLTVQARQL

SEQ ID NO: 158 - protein, 33 aa, Artificial sequence
  Peptides derived of HIV-1 gp41
AVGLGAMIFGFLGAAGSTMGAASLTLTVQARQL

SEQ ID NO: 159 - protein, 33 aa, Artificial sequence
  Peptides derived of HIV-1 gp41
AVGLGAVFFGFLGAAGSTMGAASITLTVQARQL

SEQ ID NO: 160 - protein, 33 aa, Artificial sequence
  Peptides derived of HIV-1 gp41
AVGLGAVFIGFLGAAGSTMGAASITLTVQARQL

SEQ ID NO: 161 - protein, 33 aa, Artificial sequence
  Peptides derived of HIV-1 gp41
AVGLGAVFIGFLGAAGSTMGAASMTLTVQARQL

SEQ ID NO: 162 - protein, 33 aa, Artificial sequence
  Peptides derived of HIV-1 gp41
AVGLGAVFIGFLGAAGSTMGAASVTLTVQARQL

SEQ ID NO: 163 - protein, 33 aa, Artificial sequence
  Peptides derived of HIV-1 gp41
AVGLGAVFLEFLGAAGSTMGAASVTLTVQARQL

SEQ ID NO: 164 - protein, 33 aa, Artificial sequence
  Peptides derived of HIV-1 gp41
AVGLGAVFLGFLGAAGSTMGAASITLTVQARQL

SEQ ID NO: 165 - protein, 33 aa, Artificial sequence
  Peptides derived of HIV-1 gp41
AVGLGAVFLGFLGAAGSTMGAASLTLTVQARQL

SEQ ID NO: 166 - protein, 33 aa, Artificial sequence
  Peptides derived of HIV-1 gp41
AVGLGAVFLGFLGAAGSTMGAASMTLTVQARQL

SEQ ID NO: 167 - protein, 33 aa, Artificial sequence
  Peptides derived of HIV-1 gp41
AVGLGAVFLGFLGAAGSTMGAASVTLTVQARQL

SEQ ID NO: 168 - protein, 33 aa, Artificial sequence
  Peptides derived of HIV-1 gp41
AVGLGAVFLGFLGAAGSTMGARSITLTVQARQL

SEQ ID NO: 169 - protein, 33 aa, Artificial sequence
  Peptides derived of HIV-1 gp41
AVGLGAVFLGFLGTAGSTMGAASITLTVQARQL

SEQ ID NO: 170 - protein, 33 aa, Artificial sequence
  Peptides derived of HIV-1 gp41
AVGLGAVFLGSLGAAGSTMGAASITLTVQARQL

SEQ ID NO: 171 - protein, 33 aa, Artificial sequence
  Peptides derived of HIV-1 gp41
AVGLGAVFQGFLGAAGSTMGAASITLTVQARQL

SEQ ID NO: 172 - protein, 33 aa, Artificial sequence
  Peptides derived of HIV-1 gp41
AVGLGAVIFGFLGAAGSTMGAASITLTVQARQL

SEQ ID NO: 173 - protein, 33 aa, Artificial sequence
  Peptides derived of HIV-1 gp41
AVGLGAVLFGFLGAAGSTMGAASITLTVQARQL

SEQ ID NO: 174 - protein, 33 aa, Artificial sequence
  Peptides derived of HIV-1 gp41
AVGLGAVLLGFLGAAGSTMGAASITLTVQARQL

SEQ ID NO: 175 - protein, 33 aa, Artificial sequence
  Peptides derived of HIV-1 gp41
AVGLGAVLLGFLGTAGSTMGAASITLTVQARQL

SEQ ID NO: 176 - protein, 33 aa, Artificial sequence
  Peptides derived of HIV-1 gp41
AVGLGVAFLGFLGAAGSTMGAASITLTVQARQL

SEQ ID NO: 177 - protein, 33 aa, Artificial sequence
  Peptides derived of HIV-1 gp41
AVGMAAVFFGFLGAAGSTMGAASITLTVQARQL

SEQ ID NO: 178 - protein, 33 aa, Artificial sequence
  Peptides derived of HIV-1 gp41
AVGMAAVFIGFLGAAGSTMGAASITLTVQARQL

SEQ ID NO: 179 - protein, 33 aa, Artificial sequence
  Peptides derived of HIV-1 gp41
AVGMAAVFLGFLGTAGSTMGAASLTLTVQARQL

SEQ ID NO: 180 - protein, 33 aa, Artificial sequence
  Peptides derived of HIV-1 gp41
AVGMGAFFLGFLGAAGSTMGAASITLTVQARQL

SEQ ID NO: 181 - protein, 33 aa, Artificial sequence
  Peptides derived of HIV-1 gp41
AVGMGAFFLGFLGAAGSTMGAASLTLTVQARQL

SEQ ID NO: 182 - protein, 33 aa, Artificial sequence
  Peptides derived of HIV-1 gp41
AVGMGALFLGFLGAAGSTMGAASITLTVQARQL

SEQ ID NO: 183 - protein, 33 aa, Artificial sequence
  Peptides derived of HIV-1 gp41
AVGMGALFLGFLGAAGSTMGAASLTLTVQARQL

SEQ ID NO: 184 - protein, 33 aa, Artificial sequence
  Peptides derived of HIV-1 gp41
AVGMGALFLGFLGAAGSTMGAASMTLTVQARQL

SEQ ID NO: 185 - protein, 33 aa, Artificial sequence
  Peptides derived of HIV-1 gp41
AVGMGALFLGFLGAAGSTMGAASVTLTVQARQL

SEQ ID NO: 186 - protein, 33 aa, Artificial sequence
  Peptides derived of HIV-1 gp41
AVGMGALFLGFLGAAGSTMGAVSMTLTVQARQL

SEQ ID NO: 187 - protein, 33 aa, Artificial sequence
  Peptides derived of HIV-1 gp41
AVGMGALFLGFLGTAGSTMGAVSMTLTVQARQL

SEQ ID NO: 188 - protein, 33 aa, Artificial sequence
  Peptides derived of HIV-1 gp41
AVGMGALFLGFLSAAGSTMGAASITLTVQARQL

SEQ ID NO: 189 - protein, 33 aa, Artificial sequence
  Peptides derived of HIV-1 gp41
AVGMGAMFLGFLAAAGSTMGAASLTLTVQARQL

SEQ ID NO: 190 - protein, 33 aa, Artificial sequence
  Peptides derived of HIV-1 gp41
AVGMGAMILGFLSAAGSTMGAASITLTVQARQL

SEQ ID NO: 191 - protein, 33 aa, Artificial sequence
  Peptides derived of HIV-1 gp41
AVGMGASFLGFLGAAGSTMGAASITLTVQARQL

SEQ ID NO: 192 - protein, 33 aa, Artificial sequence
  Peptides derived of HIV-1 gp41
AVGMGAVFLGFLGAAGSTMGAASITLTVQARQL

SEQ ID NO: 193 - protein, 33 aa, Artificial sequence
  Peptides derived of HIV-1 gp41
AVGMGAVFLGFLSAAGSTMGAASITLTVQARQL

SEQ ID NO: 194 - protein, 33 aa, Artificial sequence
  Peptides derived of HIV-1 gp41
AVGMGAVLLGFLGAAGSTMGAASITLTVQARQL

SEQ ID NO: 195 - protein, 33 aa, Artificial sequence
  Peptides derived of HIV-1 gp41
AVGVGALFLGFLSAAGSTMGAASITLTVQARQL

SEQ ID NO: 196 - protein, 33 aa, Artificial sequence
  Peptides derived of HIV-1 gp41
AVGVGALLIGFLGAAGSTMGAASMTLTVQARQL

SEQ ID NO: 197 - protein, 33 aa, Artificial sequence
  Peptides derived of HIV-1 gp41
AVGVGAMIFGFLGAAGSTMGAASITLTVQARQL

SEQ ID NO: 198 - protein, 33 aa, Artificial sequence
  Peptides derived of HIV-1 gp41
AVGVGAMILGFLGAAGSTMGAASITLTVQARQL

SEQ ID NO: 199 - protein, 15 aa, Artificial sequence
  Peptides derived of HIV-1 gp41
VGIGALFLGFLGAAG

SEQ ID NO: 200 - protein, 15 aa, Artificial sequence
  Peptides derived of HIV-1 gp41
VGLGAVFLGFLGAAG

SEQ ID NO: 201 - protein, 16 aa, Artificial sequence
  Peptides derived of HIV-1 gp41
AAGIGAVLLGFLGAAG

SEQ ID NO: 202 - protein, 16 aa, Artificial sequence
  Peptides derived of HIV-1 gp41
AAGIGAVLPGFLGAAG

SEQ ID NO: 203 - protein, 16 aa, Artificial sequence
  Peptides derived of HIV-1 gp41
AAGIGAVLPGFLGAAR

SEQ ID NO: 204 - protein, 16 aa, Artificial sequence
  Peptides derived of HIV-1 gp41
AAGLGAVFLGFLGAAG

SEQ ID NO: 205 - protein, 16 aa, Artificial sequence
  Peptides derived of HIV-1 gp41
AIGIGAMFLGFLGAAG

SEQ ID NO: 206 - protein, 16 aa, Artificial sequence
  Peptides derived of HIV-1 gp41
AIGIGAVFIGFLGAAG

SEQ ID NO: 207 - protein, 16 aa, Artificial sequence
  Peptides derived of HIV-1 gp41
AIGIGAVFLGFLGAAG

SEQ ID NO: 208 - protein, 16 aa, Artificial sequence
  Peptides derived of HIV-1 gp41
AIGIGAVFLGFLGTAG

SEQ ID NO: 209 - protein, 16 aa, Artificial sequence
  Peptides derived of HIV-1 gp41
AIGIGAVVLGFLGTAG

SEQ ID NO: 210 - protein, 16 aa, Artificial sequence
  Peptides derived of HIV-1 gp41
AIGLGAAFLGFLGAAG

SEQ ID NO: 211 - protein, 16 aa, Artificial sequence
  Peptides derived of HIV-1 gp41
AIGLGAALLGFLGAAG

SEQ ID NO: 212 - protein, 16 aa, Artificial sequence
  Peptides derived of HIV-1 gp41
AIGLGALFLGFLGAAG

SEQ ID NO: 213 - protein, 16 aa, Artificial sequence
  Peptides derived of HIV-1 gp41
AIGLGAMFLGFLGAAG

SEQ ID NO: 214 - protein, 16 aa, Artificial sequence
  Peptides derived of HIV-1 gp41
AIGLGAMFLGFLGAAG

SEQ ID NO: 215 - protein, 16 aa, Artificial sequence
  Peptides derived of HIV-1 gp41
AIGLGAMFLGFLGTAG

SEQ ID NO: 216 - protein, 16 aa, Artificial sequence
  Peptides derived of HIV-1 gp41
AIGLGAVFIGFLGAAG

SEQ ID NO: 217 - protein, 16 aa, Artificial sequence
  Peptides derived of HIV-1 gp41
AIGLGAVFLGFLGAAG

SEQ ID NO: 218 - protein, 16 aa, Artificial sequence
  Peptides derived of HIV-1 gp41
AIGLGAVFXGFLGAAG

SEQ ID NO: 219 - protein, 16 aa, Artificial sequence
  Peptides derived of HIV-1 gp41
AMGIGAVFLGFLGAAG

SEQ ID NO: 220 - protein, 16 aa, Artificial sequence
  Peptides derived of HIV-1 gp41
AMGIGAVLLGFLGAPG

SEQ ID NO: 221 - protein, 16 aa, Artificial sequence
  Peptides derived of HIV-1 gp41
AVGIGAALLGFLGAAG

SEQ ID NO: 222 - protein, 16 aa, Artificial sequence
  Peptides derived of HIV-1 gp41
AVGIGAFFLGFLGAAG

SEQ ID NO: 223 - protein, 16 aa, Artificial sequence
  Peptides derived of HIV-1 gp41
AVGIGAFVLGFLGAAG

SEQ ID NO: 224 - protein, 16 aa, Artificial sequence
  Peptides derived of HIV-1 gp41
AVGIGALFLGFLAAAG

SEQ ID NO: 225 - protein, 16 aa, Artificial sequence
  Peptides derived of HIV-1 gp41
AVGIGALFLGFLGAAG

SEQ ID NO: 226 - protein, 16 aa, Artificial sequence
  Peptides derived of HIV-1 gp41
AVGIGALFLGFLGPAG

SEQ ID NO: 227 - protein, 16 aa, Artificial sequence
  Peptides derived of HIV-1 gp41
AVGIGALFLGFLGTAG

SEQ ID NO: 228 - protein, 16 aa, Artificial sequence
  Peptides derived of HIV-1 gp41
AVGIGALIFGFLGAAG

SEQ ID NO: 229 - protein, 16 aa, Artificial sequence
  Peptides derived of HIV-1 gp41
AVGIGALLFGFLGAAG

SEQ ID NO: 230 - protein, 16 aa, Artificial sequence
  Peptides derived of HIV-1 gp41
AVGIGALLFGSLGAAG

SEQ ID NO: 231 - protein, 16 aa, Artificial sequence
  Peptides derived of HIV-1 gp41
AVGIGALLIGFLGAAG

SEQ ID NO: 232 - protein, 16 aa, Artificial sequence
  Peptides derived of HIV-1 gp41
AVGIGALLLGFLGAAG

SEQ ID NO: 233 - protein, 16 aa, Artificial sequence
  Peptides derived of HIV-1 gp41
AVGIGALLVGFLGAAG

SEQ ID NO: 234 - protein, 16 aa, Artificial sequence
  Peptides derived of HIV-1 gp41
AVGIGALLVGFLGTAG

SEQ ID NO: 235 - protein, 16 aa, Artificial sequence
  Peptides derived of HIV-1 gp41
AVGIGALSLGFLGAAG

SEQ ID NO: 236 - protein, 16 aa, Artificial sequence
  Peptides derived of HIV-1 gp41
AVGIGALVFGFLGAAG

SEQ ID NO: 237 - protein, 16 aa, Artificial sequence
  Peptides derived of HIV-1 gp41
AVGIGAMFLGFLGAAG

SEQ ID NO: 238 - protein, 16 aa, Artificial sequence
  Peptides derived of HIV-1 gp41
AVGIGAMFLGFLGMAG

SEQ ID NO: 239 - protein, 16 aa, Artificial sequence
  Peptides derived of HIV-1 gp41
AVGIGAMFLGILGAAG

SEQ ID NO: 240 - protein, 16 aa, Artificial sequence
  Peptides derived of HIV-1 gp41
AVGIGAMFLGILGTAG

SEQ ID NO: 241 - protein, 16 aa, Artificial sequence
  Peptides derived of HIV-1 gp41
AVGIGAMIFGFLGAAG

SEQ ID NO: 242 - protein, 16 aa, Artificial sequence
  Peptides derived of HIV-1 gp41
AVGIGAMIFGFLGAAR

SEQ ID NO: 243 - protein, 16 aa, Artificial sequence
  Peptides derived of HIV-1 gp41
AVGIGAMIFGFLGAPG

SEQ ID NO: 244 - protein, 16 aa, Artificial sequence
  Peptides derived of HIV-1 gp41
AVGIGAMIFGFSGAAG

SEQ ID NO: 245 - protein, 16 aa, Artificial sequence
  Peptides derived of HIV-1 gp41
AVGIGAMILGFLGAAG

SEQ ID NO: 246 - protein, 16 aa, Artificial sequence
  Peptides derived of HIV-1 gp41
AVGIGAMLFGFLGAAG

SEQ ID NO: 247 - protein, 16 aa, Artificial sequence
  Peptides derived of HIV-1 gp41
AVGIGAVFFGFLGAAG

SEQ ID NO: 248 - protein, 16 aa, Artificial sequence
  Peptides derived of HIV-1 gp41
AVGIGAVFIGFLGAAG

SEQ ID NO: 249 - protein, 16 aa, Artificial sequence
  Peptides derived of HIV-1 gp41
AVGIGAVFIGFLSAAG

SEQ ID NO: 250 - protein, 16 aa, Artificial sequence
  Peptides derived of HIV-1 gp41
AVGIGAVFLGFLAAAG

SEQ ID NO: 251 - protein, 16 aa, Artificial sequence
  Peptides derived of HIV-1 gp41
AVGIGAVFLGFLATAG

SEQ ID NO: 252 - protein, 16 aa, Artificial sequence
  Peptides derived of HIV-1 gp41
AVGIGAVFLGFLGAAG

SEQ ID NO: 253 - protein, 16 aa, Artificial sequence
  Peptides derived of HIV-1 gp41
AVGIGAVFLGFLGAEG

SEQ ID NO: 254 - protein, 16 aa, Artificial sequence
  Peptides derived of HIV-1 gp41
AVGIGAVFLGFLGFAG

SEQ ID NO: 255 - protein, 16 aa, Artificial sequence
  Peptides derived of HIV-1 gp41
AVGIGAVFLGFLGVAG

SEQ ID NO: 256 - protein, 16 aa, Artificial sequence
  Peptides derived of HIV-1 gp41
AVGIGAVFLGILGAAG

SEQ ID NO: 257 - protein, 16 aa, Artificial sequence
  Peptides derived of HIV-1 gp41
AVGIGAVFLGSLGAAG

SEQ ID NO: 258 - protein, 16 aa, Artificial sequence
  Peptides derived of HIV-1 gp41
AVGIGAVFLRFLGAAG

SEQ ID NO: 259 - protein, 16 aa, Artificial sequence
  Peptides derived of HIV-1 gp41
AVGIGAVFVGFLGAAG

SEQ ID NO: 260 - protein, 16 aa, Artificial sequence
  Peptides derived of HIV-1 gp41
AVGIGAVIFGFLGAAG

SEQ ID NO: 261 - protein, 16 aa, Artificial sequence
  Peptides derived of HIV-1 gp41
AVGIGAVILGFLGAAG

SEQ ID NO: 262 - protein, 16 aa, Artificial sequence
  Peptides derived of HIV-1 gp41
AVGIGAVLFGFLGAAG

SEQ ID NO: 263 - protein, 16 aa, Artificial sequence
  Peptides derived of HIV-1 gp41
AVGIGAVLIGFLGAAG

SEQ ID NO: 264 - protein, 16 aa, Artificial sequence
  Peptides derived of HIV-1 gp41
AVGIGAVLLGFLGAAG

SEQ ID NO: 265 - protein, 16 aa, Artificial sequence
  Peptides derived of HIV-1 gp41
AVGIGAVLLGFLGTAG

SEQ ID NO: 266 - protein, 16 aa, Artificial sequence
  Peptides derived of HIV-1 gp41
AVGIGAVLVGFLGAAG

SEQ ID NO: 267 - protein, 16 aa, Artificial sequence
  Peptides derived of HIV-1 gp41
AVGIGAVSLGFLGAAG

SEQ ID NO: 268 - protein, 16 aa, Artificial sequence
  Peptides derived of HIV-1 gp41
AVGIGIMIFGFLGAAG

SEQ ID NO: 269 - protein, 16 aa, Artificial sequence
  Peptides derived of HIV-1 gp41
AVGIGIMIFGFLGAAR

SEQ ID NO: 270 - protein, 16 aa, Artificial sequence
  Peptides derived of HIV-1 gp41
AVGIGTMIFGFLGAAG

SEQ ID NO: 271 - protein, 16 aa, Artificial sequence
  Peptides derived of HIV-1 gp41
AVGIGVLLLGFLGAAG

SEQ ID NO: 272 - protein, 16 aa, Artificial sequence
  Peptides derived of HIV-1 gp41
AVGIGVVFFGFLGAAG

SEQ ID NO: 273 - protein, 16 aa, Artificial sequence
  Peptides derived of HIV-1 gp41
AVGLAAVFFGFLGAAG

SEQ ID NO: 274 - protein, 16 aa, Artificial sequence
  Peptides derived of HIV-1 gp41
AVGLEAVFLGFLGAAG

SEQ ID NO: 275 - protein, 16 aa, Artificial sequence
  Peptides derived of HIV-1 gp41
AVGLGAAFLGFLGAAG

SEQ ID NO: 276 - protein, 16 aa, Artificial sequence
  Peptides derived of HIV-1 gp41
AVGLGAFFLGFLGAAG

SEQ ID NO: 277 - protein, 16 aa, Artificial sequence
  Peptides derived of HIV-1 gp41
AVGLGAFFLGFLGVAG

SEQ ID NO: 278 - protein, 16 aa, Artificial sequence
  Peptides derived of HIV-1 gp41
AVGLGAIFIGFLGAAG

SEQ ID NO: 279 - protein, 16 aa, Artificial sequence
  Peptides derived of HIV-1 gp41
AVGLGALFFGFLGAAG

SEQ ID NO: 280 - protein, 16 aa, Artificial sequence
  Peptides derived of HIV-1 gp41
AVGLGALFIGFLGAAG

SEQ ID NO: 281 - protein, 16 aa, Artificial sequence
  Peptides derived of HIV-1 gp41
AVGLGALFLGFLGAAG

SEQ ID NO: 282 - protein, 16 aa, Artificial sequence
  Peptides derived of HIV-1 gp41
AVGLGALFLGFLGGAG

SEQ ID NO: 283 - protein, 16 aa, Artificial sequence
  Peptides derived of HIV-1 gp41
AVGLGALLFGFLGAAG

SEQ ID NO: 284 - protein, 16 aa, Artificial sequence
  Peptides derived of HIV-1 gp41
AVGLGAMFIGFLGAAG

SEQ ID NO: 285 - protein, 16 aa, Artificial sequence
  Peptides derived of HIV-1 gp41
AVGLGAMFLGFLGAAG

SEQ ID NO: 286 - protein, 16 aa, Artificial sequence
  Peptides derived of HIV-1 gp41
AVGLGAMIFGFLGAAG

SEQ ID NO: 287 - protein, 16 aa, Artificial sequence
  Peptides derived of HIV-1 gp41
AVGLGAVFFGFLGAAG

SEQ ID NO: 288 - protein, 16 aa, Artificial sequence
  Peptides derived of HIV-1 gp41
AVGLGAVFIGFLGAAG

SEQ ID NO: 289 - protein, 16 aa, Artificial sequence
  Peptides derived of HIV-1 gp41
AVGLGAVFLEFLGAAG

SEQ ID NO: 290 - protein, 16 aa, Artificial sequence
  Peptides derived of HIV-1 gp41
AVGLGAVFLGFLGAAG

SEQ ID NO: 291 - protein, 16 aa, Artificial sequence
  Peptides derived of HIV-1 gp41
AVGLGAVFLGFLGTAG

SEQ ID NO: 292 - protein, 16 aa, Artificial sequence
  Peptides derived of HIV-1 gp41
AVGLGAVFLGSLGAAG

SEQ ID NO: 293 - protein, 16 aa, Artificial sequence
  Peptides derived of HIV-1 gp41
AVGLGAVFQGFLGAAG

SEQ ID NO: 294 - protein, 16 aa, Artificial sequence
  Peptides derived of HIV-1 gp41
AVGLGAVIFGFLGAAG

SEQ ID NO: 295 - protein, 16 aa, Artificial sequence
  Peptides derived of HIV-1 gp41
AVGLGAVLFGFLGAAG

SEQ ID NO: 296 - protein, 16 aa, Artificial sequence
  Peptides derived of HIV-1 gp41
AVGLGAVLLGFLGAAG

SEQ ID NO: 297 - protein, 16 aa, Artificial sequence
  Peptides derived of HIV-1 gp41
AVGLGAVLLGFLGTAG

SEQ ID NO: 298 - protein, 16 aa, Artificial sequence
  Peptides derived of HIV-1 gp41
AVGLGVAFLGFLGAAG

SEQ ID NO: 299 - protein, 16 aa, Artificial sequence
  Peptides derived of HIV-1 gp41
AVGMAAVFFGFLGAAG

SEQ ID NO: 300 - protein, 16 aa, Artificial sequence
  Peptides derived of HIV-1 gp41
AVGMAAVFIGFLGAAG

SEQ ID NO: 301 - protein, 16 aa, Artificial sequence
  Peptides derived of HIV-1 gp41
AVGMAAVFLGFLGTAG

SEQ ID NO: 302 - protein, 16 aa, Artificial sequence
  Peptides derived of HIV-1 gp41
AVGMGAFFLGFLGAAG

SEQ ID NO: 303 - protein, 16 aa, Artificial sequence
  Peptides derived of HIV-1 gp41
AVGMGALFLGFLGAAG

SEQ ID NO: 304 - protein, 16 aa, Artificial sequence
  Peptides derived of HIV-1 gp41
AVGMGALFLGFLGTAG

SEQ ID NO: 305 - protein, 16 aa, Artificial sequence
  Peptides derived of HIV-1 gp41
AVGMGALFLGFLSAAG

SEQ ID NO: 306 - protein, 16 aa, Artificial sequence
  Peptides derived of HIV-1 gp41
AVGMGAMFLGFLAAAG

SEQ ID NO: 307 - protein, 16 aa, Artificial sequence
  Peptides derived of HIV-1 gp41
AVGMGAMILGFLSAAG

SEQ ID NO: 308 - protein, 16 aa, Artificial sequence
  Peptides derived of HIV-1 gp41
AVGMGASFLGFLGAAG

SEQ ID NO: 309 - protein, 16 aa, Artificial sequence
  Peptides derived of HIV-1 gp41
AVGMGAVFLGFLGAAG

SEQ ID NO: 310 - protein, 16 aa, Artificial sequence
  Peptides derived of HIV-1 gp41
AVGMGAVFLGFLSAAG

SEQ ID NO: 311 - protein, 16 aa, Artificial sequence
  Peptides derived of HIV-1 gp41
AVGMGAVLLGFLGAAG

SEQ ID NO: 312 - protein, 16 aa, Artificial sequence
  Peptides derived of HIV-1 gp41
AVGVGALFLGFLSAAG

SEQ ID NO: 313 - protein, 16 aa, Artificial sequence
  Peptides derived of HIV-1 gp41
AVGVGALLIGFLGAAG

SEQ ID NO: 314 - protein, 16 aa, Artificial sequence
  Peptides derived of HIV-1 gp41
AVGVGAMIFGFLGAAG

SEQ ID NO: 315 - protein, 16 aa, Artificial sequence
  Peptides derived of HIV-1 gp41
AVGVGAMILGFLGAAG

SEQ ID NO: 316 - protein, 11 aa, Artificial sequence
  Peptides derived of HIV-1 gp41
VGIGALFLGFL

SEQ ID NO: 317 - protein, 11 aa, Artificial sequence
  Peptides derived of HIV-1 gp41
VGLGAVFLGFL

SEQ ID NO: 318 - protein, 12 aa, Artificial sequence
  Peptides derived of HIV-1 gp41
AAGIGAVLLGFL

SEQ ID NO: 319 - protein, 12 aa, Artificial sequence
  Peptides derived of HIV-1 gp41
AAGIGAVLPGFL

SEQ ID NO: 320 - protein, 12 aa, Artificial sequence
  Peptides derived of HIV-1 gp41
AAGLGAVFLGFL

SEQ ID NO: 321 - protein, 12 aa, Artificial sequence
  Peptides derived of HIV-1 gp41
AIGIGAMFLGFL

SEQ ID NO: 322 - protein, 12 aa, Artificial sequence
  Peptides derived of HIV-1 gp41
AIGIGAVFIGFL

SEQ ID NO: 323 - protein, 12 aa, Artificial sequence
  Peptides derived of HIV-1 gp41
AIGIGAVFLGFL

SEQ ID NO: 324 - protein, 12 aa, Artificial sequence
  Peptides derived of HIV-1 gp41
AIGIGAVVLGFL

SEQ ID NO: 325 - protein, 12 aa, Artificial sequence
  Peptides derived of HIV-1 gp41
AIGLGAAFLGFL

SEQ ID NO: 326 - protein, 12 aa, Artificial sequence
  Peptides derived of HIV-1 gp41
AIGLGAALLGFL

SEQ ID NO: 327 - protein, 12 aa, Artificial sequence
  Peptides derived of HIV-1 gp41
AIGLGALFLGFL

SEQ ID NO: 328 - protein, 12 aa, Artificial sequence
  Peptides derived of HIV-1 gp41
AIGLGAMFLGFL

SEQ ID NO: 329 - protein, 12 aa, Artificial sequence
  Peptides derived of HIV-1 gp41
AIGLGAVFIGFL

SEQ ID NO: 330 - protein, 12 aa, Artificial sequence
  Peptides derived of HIV-1 gp41
AIGLGAVFLGFL

SEQ ID NO: 331 - protein, 12 aa, Artificial sequence
  Peptides derived of HIV-1 gp41
AIGLGAVFXGFL

SEQ ID NO: 332 - protein, 12 aa, Artificial sequence
  Peptides derived of HIV-1 gp41
AMGIGAVFLGFL

SEQ ID NO: 333 - protein, 12 aa, Artificial sequence
  Peptides derived of HIV-1 gp41
AMGIGAVLLGFL

SEQ ID NO: 334 - protein, 12 aa, Artificial sequence
  Peptides derived of HIV-1 gp41
AVGIGAALLGFL

SEQ ID NO: 335 - protein, 12 aa, Artificial sequence
  Peptides derived of HIV-1 gp41
AVGIGAFFLGFL

SEQ ID NO: 336 - protein, 12 aa, Artificial sequence
  Peptides derived of HIV-1 gp41
AVGIGAFVLGFL

SEQ ID NO: 337 - protein, 12 aa, Artificial sequence
  Peptides derived of HIV-1 gp41
AVGIGALFLGFL

SEQ ID NO: 338 - protein, 12 aa, Artificial sequence
  Peptides derived of HIV-1 gp41
AVGIGALIFGFL

SEQ ID NO: 339 - protein, 12 aa, Artificial sequence
  Peptides derived of HIV-1 gp41
AVGIGALLFGFL

SEQ ID NO: 340 - protein, 12 aa, Artificial sequence
  Peptides derived of HIV-1 gp41
AVGIGALLFGSL

SEQ ID NO: 341 - protein, 12 aa, Artificial sequence
  Peptides derived of HIV-1 gp41
AVGIGALLIGFL

SEQ ID NO: 342 - protein, 12 aa, Artificial sequence
  Peptides derived of HIV-1 gp41
AVGIGALLLGFL

SEQ ID NO: 343 - protein, 12 aa, Artificial sequence
  Peptides derived of HIV-1 gp41
AVGIGALLVGFL

SEQ ID NO: 344 - protein, 12 aa, Artificial sequence
  Peptides derived of HIV-1 gp41
AVGIGALSLGFL

SEQ ID NO: 345 - protein, 12 aa, Artificial sequence
  Peptides derived of HIV-1 gp41
AVGIGALVFGFL

SEQ ID NO: 346 - protein, 12 aa, Artificial sequence
  Peptides derived of HIV-1 gp41
AVGIGAMFLGFL

SEQ ID NO: 347 - protein, 12 aa, Artificial sequence
  Peptides derived of HIV-1 gp41
AVGIGAMFLGIL

SEQ ID NO: 348 - protein, 12 aa, Artificial sequence
  Peptides derived of HIV-1 gp41
AVGIGAMIFGFL

SEQ ID NO: 349 - protein, 12 aa, Artificial sequence
  Peptides derived of HIV-1 gp41
AVGIGAMIFGFS

SEQ ID NO: 350 - protein, 12 aa, Artificial sequence
  Peptides derived of HIV-1 gp41
AVGIGAMILGFL

SEQ ID NO: 351 - protein, 12 aa, Artificial sequence
  Peptides derived of HIV-1 gp41
AVGIGAMLFGFL

SEQ ID NO: 352 - protein, 12 aa, Artificial sequence
  Peptides derived of HIV-1 gp41
AVGIGAVFFGFL

SEQ ID NO: 353 - protein, 12 aa, Artificial sequence
  Peptides derived of HIV-1 gp41
AVGIGAVFIGFL

SEQ ID NO: 354 - protein, 12 aa, Artificial sequence
  Peptides derived of HIV-1 gp41
AVGIGAVFLGFL

SEQ ID NO: 355 - protein, 12 aa, Artificial sequence
  Peptides derived of HIV-1 gp41
AVGIGAVFLGIL

SEQ ID NO: 356 - protein, 12 aa, Artificial sequence
  Peptides derived of HIV-1 gp41
AVGIGAVFLGSL

SEQ ID NO: 357 - protein, 12 aa, Artificial sequence
  Peptides derived of HIV-1 gp41
AVGIGAVFLRFL

SEQ ID NO: 358 - protein, 12 aa, Artificial sequence
  Peptides derived of HIV-1 gp41
AVGIGAVFVGFL

SEQ ID NO: 359 - protein, 12 aa, Artificial sequence
  Peptides derived of HIV-1 gp41
AVGIGAVIFGFL

SEQ ID NO: 360 - protein, 12 aa, Artificial sequence
  Peptides derived of HIV-1 gp41
AVGIGAVILGFL

SEQ ID NO: 361 - protein, 12 aa, Artificial sequence
  Peptides derived of HIV-1 gp41
AVGIGAVLFGFL

SEQ ID NO: 362 - protein, 12 aa, Artificial sequence
  Peptides derived of HIV-1 gp41
AVGIGAVLIGFL

SEQ ID NO: 363 - protein, 12 aa, Artificial sequence
  Peptides derived of HIV-1 gp41
AVGIGAVLLGFL

SEQ ID NO: 364 - protein, 12 aa, Artificial sequence
  Peptides derived of HIV-1 gp41
AVGIGAVLVGFL

SEQ ID NO: 365 - protein, 12 aa, Artificial sequence
  Peptides derived of HIV-1 gp41
AVGIGAVSLGFL

SEQ ID NO: 366 - protein, 12 aa, Artificial sequence
  Peptides derived of HIV-1 gp41
AVGIGIMIFGFL

SEQ ID NO: 367 - protein, 12 aa, Artificial sequence
  Peptides derived of HIV-1 gp41
AVGIGTMIFGFL

SEQ ID NO: 368 - protein, 12 aa, Artificial sequence
  Peptides derived of HIV-1 gp41
AVGIGVLLLGFL

SEQ ID NO: 369 - protein, 12 aa, Artificial sequence
  Peptides derived of HIV-1 gp41
AVGIGVVFFGFL

SEQ ID NO: 370 - protein, 12 aa, Artificial sequence
  Peptides derived of HIV-1 gp41
AVGLAAVFFGFL

SEQ ID NO: 371 - protein, 12 aa, Artificial sequence
  Peptides derived of HIV-1 gp41
AVGLEAVFLGFL

SEQ ID NO: 372 - protein, 12 aa, Artificial sequence
  Peptides derived of HIV-1 gp41
AVGLGAAFLGFL

SEQ ID NO: 373 - protein, 12 aa, Artificial sequence
  Peptides derived of HIV-1 gp41
AVGLGAFFLGFL

SEQ ID NO: 374 - protein, 12 aa, Artificial sequence
  Peptides derived of HIV-1 gp41
AVGLGAIFIGFL

SEQ ID NO: 375 - protein, 12 aa, Artificial sequence
  Peptides derived of HIV-1 gp41
AVGLGALFFGFL

SEQ ID NO: 376 - protein, 12 aa, Artificial sequence
  Peptides derived of HIV-1 gp41
AVGLGALFIGFL

SEQ ID NO: 377 - protein, 12 aa, Artificial sequence
  Peptides derived of HIV-1 gp41
AVGLGALFLGFL

SEQ ID NO: 378 - protein, 12 aa, Artificial sequence
  Peptides derived of HIV-1 gp41
AVGLGALLFGFL

SEQ ID NO: 379 - protein, 12 aa, Artificial sequence
  Peptides derived of HIV-1 gp41
AVGLGAMFIGFL

SEQ ID NO: 380 - protein, 12 aa, Artificial sequence
  Peptides derived of HIV-1 gp41
AVGLGAMFLGFL

SEQ ID NO: 381 - protein, 12 aa, Artificial sequence
  Peptides derived of HIV-1 gp41
AVGLGAMIFGFL

SEQ ID NO: 382 - protein, 12 aa, Artificial sequence
  Peptides derived of HIV-1 gp41
AVGLGAVFFGFL

SEQ ID NO: 383 - protein, 12 aa, Artificial sequence
  Peptides derived of HIV-1 gp41
AVGLGAVFIGFL

SEQ ID NO: 384 - protein, 12 aa, Artificial sequence
  Peptides derived of HIV-1 gp41
AVGLGAVFLEFL

SEQ ID NO: 385 - protein, 12 aa, Artificial sequence
  Peptides derived of HIV-1 gp41
AVGLGAVFLGFL

SEQ ID NO: 386 - protein, 12 aa, Artificial sequence
  Peptides derived of HIV-1 gp41
AVGLGAVFLGSL

SEQ ID NO: 387 - protein, 12 aa, Artificial sequence
  Peptides derived of HIV-1 gp41
AVGLGAVFQGFL

SEQ ID NO: 388 - protein, 12 aa, Artificial sequence
  Peptides derived of HIV-1 gp41
AVGLGAVIFGFL

SEQ ID NO: 389 - protein, 12 aa, Artificial sequence
  Peptides derived of HIV-1 gp41
AVGLGAVLFGFL

SEQ ID NO: 390 - protein, 12 aa, Artificial sequence
  Peptides derived of HIV-1 gp41
AVGLGAVLLGFL

SEQ ID NO: 391 - protein, 12 aa, Artificial sequence
  Peptides derived of HIV-1 gp41
AVGLGVAFLGFL

SEQ ID NO: 392 - protein, 12 aa, Artificial sequence
  Peptides derived of HIV-1 gp41
AVGMAAVFFGFL

SEQ ID NO: 393 - protein, 12 aa, Artificial sequence
  Peptides derived of HIV-1 gp41
AVGMAAVFIGFL

SEQ ID NO: 394 - protein, 12 aa, Artificial sequence
  Peptides derived of HIV-1 gp41
AVGMAAVFLGFL

SEQ ID NO: 395 - protein, 12 aa, Artificial sequence
  Peptides derived of HIV-1 gp41
AVGMGAFFLGFL

SEQ ID NO: 396 - protein, 12 aa, Artificial sequence
  Peptides derived of HIV-1 gp41
AVGMGALFLGFL

SEQ ID NO: 397 - protein, 12 aa, Artificial sequence
  Peptides derived of HIV-1 gp41
AVGMGAMFLGFL

SEQ ID NO: 398 - protein, 12 aa, Artificial sequence
  Peptides derived of HIV-1 gp41
AVGMGAMILGFL

SEQ ID NO: 399 - protein, 12 aa, Artificial sequence
  Peptides derived of HIV-1 gp41
AVGMGASFLGFL

SEQ ID NO: 400 - protein, 12 aa, Artificial sequence
  Peptides derived of HIV-1 gp41
AVGMGAVFLGFL

SEQ ID NO: 401 - protein, 12 aa, Artificial sequence
  Peptides derived of HIV-1 gp41
AVGMGAVLLGFL

SEQ ID NO: 402 - protein, 12 aa, Artificial sequence
  Peptides derived of HIV-1 gp41
AVGVGALFLGFL

SEQ ID NO: 403 - protein, 12 aa, Artificial sequence
  Peptides derived of HIV-1 gp41
AVGVGALLIGFL

SEQ ID NO: 404 - protein, 12 aa, Artificial sequence
  Peptides derived of HIV-1 gp41
AVGVGAMIFGFL

SEQ ID NO: 405 - protein, 12 aa, Artificial sequence
  Peptides derived of HIV-1 gp41
AVGVGAMILGFL

SEQ ID NO: 406 - protein, 8 aa, Artificial sequence
  Peptides derived of HIV-1 gp41
AVGIGALF

SEQ ID NO: 407 - protein, 9 aa, Artificial sequence
  Peptides derived of HIV-1 gp41
GALFLGFLG

SEQ ID NO: 408 - protein, 9 aa, Artificial sequence
  synthetic peptide
GALFLGFLG

SEQ ID NO: 409 - protein, 9 aa, Artificial sequence
  Peptides derived of HIV-1 gp41
GAVFLGFLG

SEQ ID NO: 410 - protein, 9 aa, Artificial sequence
  Peptides derived of HIV-1 gp41
GAMFLGFLG

SEQ ID NO: 411 - protein, 9 aa, Artificial sequence
  Peptides derived of HIV-1 gp41
GAVLLGFLG

SEQ ID NO: 412 - protein, 9 aa, Artificial sequence
  Peptides derived of HIV-1 gp41
GAFFLGFLG

SEQ ID NO: 413 - protein, 9 aa, Artificial sequence
  Peptides derived of HIV-1 gp41
GAMIFGFLG

SEQ ID NO: 414 - protein, 9 aa, Artificial sequence
  Peptides derived of HIV-1 gp41
GALLFGFLG